(19) United States
(12) Patent Application Publication
(10) Pub. No.: US 2020/0411136 A1
(43) Pub. Date: Dec. 31, 2020

(54) DETERMINING IMPACT ON PROPERTIES OF PROTEINS BASED ON AMINO ACID SEQUENCE MODIFICATIONS

(71) Applicant: Just Biotherapeutics, Inc., Seattle, WA (US)

(72) Inventor: Jeremy Martin Shaver, Lake Forest Park, WA (US)

(21) Appl. No.: 16/975,916

(22) PCT Filed: Feb. 26, 2019
(86) PCT No.: PCT/US2019/019530

§ 371 Date: Aug. 26, 2020

Related U.S. Application Data
(60) Provisional application No. 62/635,536, filed on Feb. 26, 2018.

(51) Int. Cl. G16B 20/50; G16B 30/10

(52) U.S. Cl. CPC G16B 20/50; G16B 30/10

(57) ABSTRACT

Technologies are described related to determining the impact of substitutions of amino acid sequences of proteins on properties of the base protein. Values of properties for proteins that include a particular substitution are analyzed with respect to values of properties for proteins that do not include the particular substitution. The analysis can be utilized to determine the impact of the particular substitution on the properties of the proteins while minimizing the number of proteins that need to be expressed. The impact of the particular substitution on the proteins can indicate changes to the stability and/or yield of the proteins.

### CROSS-REFERENCE TO RELATED APPLICATION(S)

This application is claims priority to U.S. Provisional Application No. 62/635,536 filed on Feb. 26, 2018 and entitled “Determining Impact on Properties of Proteins Based on Amino Acid Sequence Modifications,” the entirety of which is incorporated herein by reference.

### BACKGROUND

Proteins are comprised of a sequence of amino acids that are linked via chemical bonds. The amino acid sequence of a particular protein is based on a sequence of nucleotides in the deoxyribonucleic acid (DNA) from which the protein is expressed. The functionality and structure of a protein can be based on the amino acid sequence of the protein. Proteins can have a variety of functions within an organism, such as regulation of enzymatic activity or cellular signaling. Some proteins can also be used therapeutically to treat a biological condition. For example, proteins, such as an antibody, can bind to a pathogen to target the pathogen for destruction by other agents in the organism, such as T cells or macrophages. In another example, proteins can bind to a molecule to transport the molecule to a targeted location in an organism to alleviate phenotypes of a biological condition.

The effectiveness and viability of utilizing proteins therapeutically can depend on the stability of the proteins under certain environmental conditions. In some cases, the functionality of proteins can degrade as temperatures increase in an environment of the protein. To illustrate, proteins can unfold in response to exposure to certain temperatures, which results in a loss of the ability of the proteins to bind their target molecules. Additionally, the expression of some proteins can be costly, especially in the face of low yields. In certain scenarios, the yield for proteins can depend on the robustness of the protein in relation to environmental conditions in which the protein is expressed. For example, the yield of some proteins can decrease as the pH decreases in the environment to which the proteins are exposed.

### BRIEF DESCRIPTION OF THE DRAWINGS

FIG. 1 is a diagram of some implementations of an architecture to determine the impact of substitutions to an amino acid sequence of a base protein on the properties of proteins that are variants of the base protein.

FIG. 2 illustrates some implementations of techniques to couple a modified protein having a candidate substitution to an amino acid sequence of a base protein with a single modified protein that does not include the candidate substitution.

FIG. 3 illustrates some implementations of techniques to couple a modified protein having a candidate substitution to an amino acid sequence of a base protein with multiple modified proteins that do not include the candidate substitution.

FIGS. 4A and 4B illustrate some implementations of techniques to analyze differences between values of properties of modified proteins that have a candidate substitution to an amino acid sequence of a base protein with respect to values of properties of modified proteins that do not have the candidate substitution.

FIG. 5 illustrates some implementations of a system to determine the impact on properties of proteins based on modifications to amino acid sequences of the proteins.

FIG. 6 is a flow diagram of a first example process to identify properties of proteins that are impacted by substitutions of amino acid sequences of the proteins.

FIG. 7 is a flow diagram of a second example process to identify properties of proteins that are impacted by substitutions of amino acid sequences of the proteins.

FIG. 8A illustrates a first plot of values of properties of proteins that have been modified to include a particular substitution with respect to a base protein and values of properties of proteins that have not been modified to include the particular substitution and FIG. 8B illustrates a second plot showing couplings between proteins that have been modified to include the particular substitution and proteins that have not been modified to include the particular substitution.

FIG. 9 illustrates a plot that shows the data points of the first plot of FIG. 8 modified based on the couplings shown in the second plot of FIG. 8.

FIG. 10 illustrates a first plot showing a difference in means between a first group of data derived from proteins that have a first candidate substitution and a second group of data derived from proteins that do not have the first candidate substitution and a second plot showing a difference in means between a first group of data derived from proteins that have a second candidate substitution and a second group of data derived from proteins that do not have the second candidate substitution.

### DETAILED DESCRIPTION

The concepts described herein are directed to determining the impact on properties of proteins based on modifications to amino acid sequences of the proteins. In some instances, the modifications to the amino acid sequences of the proteins can improve the stability of the proteins and the yield for the expression of the proteins. In particular, implementations, the temperature at which a protein unfolds can be increased by making modifications to particular amino acids included in the sequence of the proteins. In additional situations, the pH at which the proteins begin to degrade can be lowered when certain modifications are made to amino acids in the sequence of the proteins.

It can often be difficult to identify amino acid residues within a protein sequence that can be modified to improve properties of a protein. In particular, each protein sequence can include hundreds, up to thousands of amino acids and identifying the particular amino acids to modify that result in improvements to the properties of the proteins can be a time intensive and resource intensive process. Furthermore, identifying which substitution to make at a particular position can also be a time intensive and resource intensive process. That is, determining which amino acid to use to replace a current amino acid at a specified position can be a time and resource intensive process. Additionally, the effects of some changes to an amino acid sequence can be difficult to predict because changes to amino acid sequences of proteins can sometimes have unintended results. (See Eriksson A E, Baase W A, Zhang X J, Heinz D W, Blaber M, Baldwin E P, Matthews B W: Response of a protein structure to cavity-creating mutations and its relation to the hydrophobic effect. Science. 1992, 255 (5041): 178-183. 10.1126/science.1553543).

Typically, to determine whether a modification to an amino acid sequence of a protein has an effect on certain properties of the protein, the DNA utilized to express the protein is changed to encode for the modified amino acid sequence at a certain position and then the protein can be expressed through the translation of the modified DNA. Thus, each time that a modification to a position of a protein sequence is to be explored to determine the effect of the modification, DNA encoding the new proteins needs to be synthesized, the modified protein needs to be expressed, and then the properties of the modified protein can be tested. The properties of the modified protein can be compared to the properties of the unmodified protein to determine whether or not the modification of the amino acid at a particular position has an impact on the properties of the protein. Accordingly, when substitutions at multiple positions for multiple different amino acid substitutions are to be analyzed, several hundred, if not thousands of protein molecules would need to be expressed and tested to determine the effects of the modifications with respect to the properties of the unmodified protein.

Some conventional techniques may attempt to predict the effect of certain substitutions on an amino acid sequence of a protein by analyzing a balanced data set. In a balanced data set, for each protein that is expressed having a particular substitution, at least one or more additional proteins are expressed that do not include the particular substitution. The balanced data set can be utilized to determine whether modifications to an amino acid sequence would produce a statistically significant effect on certain properties of the protein. By producing a balanced data set, any changes brought about by the substitution at a single position can be directly attributed to the substitution. However, the use of a balanced data set can be impractical in some situations when many substitutions are to be evaluated and the number of proteins that needs to be expressed to produce the balanced data set is relatively large, such as on the order of several hundred or more proteins. Thus, the amount of materials needed to express the proteins, such as the expression medium, vectors, host cells, etc., can be cost prohibitive. In the alternative, the number of modifications to the protein may be smaller, but this approach limits the possibilities for determining amino acid sequence modifications that impact the properties of the protein.

The techniques described herein utilize unbalanced data sets to identify residues of a base protein that can be modified to improve the yield of modified proteins and/or improve the stability of modified proteins in relation to the base protein. As used herein, a “base protein” refers to a protein that has not undergone any changes to its amino acid sequence that are produced by modifications made to the DNA sequence of the protein by a human and/or machine in a laboratory environment. In particular implementations, a group of proteins can be expressed that include modifications with respect to the amino acid sequence of a base protein. That is, the proteins included in the group of proteins can have amino acids located at one or more positions that have been modified with respect to the amino acids at the same position in a base protein. For example, a base protein can have an alanine at a specified position, while a modified protein can have a guanine at the same position. In certain cases, the amino acid sequences of the modified proteins can be different at a plurality of positions in relation to the amino acid sequence of the base protein. The modifications to the base protein are made by modifying a DNA sequence of the base protein and then expressing the modified proteins utilizing the modified DNA sequence.

To determine the effect of making a substitution at a particular position of an amino acid sequence of a protein, a first set of proteins can be expressed that have the substitution and a second set of proteins can be expressed that do not have the substitution. There can also be other differences between the amino acid sequences of the first set of proteins and the second set of proteins such that multiple substitutions can be evaluated. For each protein where the particular substitution was made, one or more additional proteins where the substitution was not made can be identified and associated with a corresponding protein for which the substitution was made. In particular implementations, the amino acid sequences of the proteins that include a particular substitution can have multiple differences with the amino acid sequences of the proteins that do not have the particular substitution. In these situations, individual proteins that do have a particular substitution can be coupled with one or more proteins that do not have the particular substitution in such a way that minimizes the number of differences between the amino acid sequence of the individual proteins having the particular substitution and the amino acid sequences of the proteins not having the particular substitution. In certain situations, multiple proteins that do not have a substitution can be coupled to a single protein that does include the substitution.

After the associations are made between the protein with the particular substitution and the one or more proteins for which the substitution was not made, differences between values of one or more properties of the proteins can be determined. In implementations where a single protein with a particular substitution is coupled with multiple proteins that do not have the substitution, the values for a property of the multiple proteins without the substitution can be combined into a single value, such as by determining an average of the values of the property for the multiple proteins without the substitution. In these situations, the single value of the property for the multiple proteins without the substitution can then be utilized to determine the difference with the value of the property having the substitution. In this way, the techniques described herein can compensate for the lack of a balanced data set. Thus, through implementing the techniques described herein, an unbalanced data set can be utilized to accurately determine whether a substitution of interest has an effect on properties of proteins. Additionally, by utilizing an unbalanced data set instead of a balanced data set, the number of proteins that need to be expressed in order to determine whether a number of substitutions that have an effect on the properties of the proteins is reduced. Reducing the number of proteins expressed results in fewer resources being utilized in order to identify substitutions to amino acid sequences that have an influence on properties of the proteins.

In some cases, the properties being analyzed can be related to the stability of the proteins. For example, the difference between a temperature at which a protein having a particular substitution unfolds and the temperatures at which the one or more proteins that do not have the substitution unfold can be determined. In another example, the difference between Gibbs free energy of a protein having a particular substitution and one or more proteins that do not have the substitution can be determined. Additionally, the properties being evaluated can be related to improving yield of the proteins that include a particular substitution with respect to proteins that do not include the substitution. Based at least partly on the differences between the values of the properties of the proteins having a particular substitution and proteins not having the substitution, a determination can be made as to whether the substitution has an effect on the particular properties. In various implementations, an analysis can be performed to determine whether differences between values of properties for proteins having a particular substitution and values of properties for proteins not having the substitution are statistically significant. By correlating changes in certain physical properties of proteins based on substitutions to particular positions of the amino acid sequence, changes to a base protein can be made that can lead to a more stable protein and/or a protein that can be produced at higher yields. In some cases, the modified protein can have the same or similar functionality as the base protein with some improved properties. Increased stability for the protein can cause the protein to remain viable under conditions where the base protein would not be viable. Accordingly, the modified protein can be transported and/or stored under conditions that the base protein could not be. This can increase the number of subjects that can receive treatment utilizing the modified protein than the base protein because some subjects may reside in areas where refrigeration is unavailable or in areas with climates that may not be favorable for the base protein. Additionally, modifying particular physical properties of a base protein through substitutions to positions of the amino acid sequence of the base protein can provide increased yields for the modified protein with respect to the base protein. That is, the physical properties of the modified protein cause it to be more robust during the expression of the modified protein, which can lead to more of the modified protein being produced than the base protein under the same conditions. In this way, the cost of manufacturing the protein can decrease.

In the following detailed description, references are made to the accompanying drawings that form a part hereof, and that show, by way of illustration, specific configurations or examples. The drawings herein are not drawn to scale. Like numerals represent like elements throughout the several figures (which might be referred to herein as a “FIG.” or “FIGS.”).

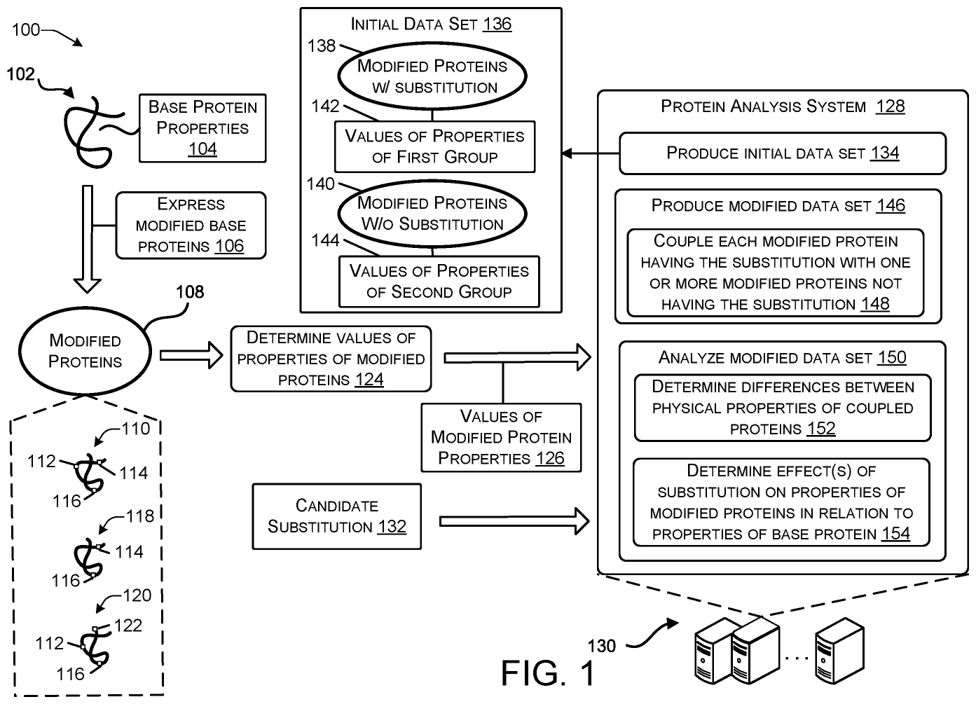

FIG. 1 is a diagram of some implementations of an architecture 100 to determine the impact of substitutions to an amino acid sequence of a base protein on the properties of proteins that are variants of the base protein. In particular, the architecture 100 is directed to identifying substitutions of amino acids at particular positions of a base protein 102 and determining whether the substitutions influence certain physical properties of the modified proteins in relation to the properties of the base protein 102. The base protein 102 can comprise a sequence of amino acids that are chemically bonded. In some cases, the amino acids comprising the base protein 102 can be coupled to each other via peptide bonds.

The base protein 102 can have secondary structure that forms between different portions of the amino acid sequence. Some examples of secondary structure of the base protein 102 can include an α-helix or a β-sheet. The base protein 102 can also include one or more turns and/or one or more loops. Additionally, the base protein 102 can have a tertiary structure that is produced by folding of the base protein 102. The tertiary structure of the base protein 102 can be a 3-dimensional structure.

The base protein 102 can have biological functionality. The biological functionality of the base protein 102 can be based at least partly on the amino acid sequence of the base protein 102 and/or the 3-dimensional structure of the base protein 102. In some particular examples, the base protein 102 can function as an enzyme. Enzymes can cause one or more chemical reactions to take place within an organism. In various situations, an enzyme can be a catalyst to cause a biochemical reaction to occur within an organism. In certain scenarios, an enzyme can bind to another molecule to cause a chemical reaction to proceed. Further, an enzyme can bind to a molecule and modify the molecule to produce a product, where the product can participate in a chemical reaction. More information regarding the functionality of enzymes can be found in Martinez Cuesta S, Rahman S A, Furnham N, Thornton J M. The Classification and Evolution of Enzyme Function. Biophysical Journal. 2015; 109(6):1082-1086. doi:10.1016/j.bpj 0.2015.04.020, which is incorporated by reference herein in its entirety.

In other examples, the base protein 102 can function as an antibody. An antibody can bind to molecules that produce a response by the immune system of an organism. The molecules bound by an antibody can include antigens. The term “antibody” is used in the broadest sense and can include fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)2, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Examples of antibody fragments can include Antibodies of any isotype class or subclass, including IgG, IgM, IgD, IgA, and IgE, IgG1, IgG2, IgG3, IgG4, IgA1 and IgA2, or any allotype, are contemplated.

An antibody can have a Y-shaped structure and includes two heavy chains and two light chains. The heavy chains include more amino acids than the light chains. In some cases, the heavy chains can each include a variable region coupled to a first constant region that is coupled to a hinge region. The hinge region is then coupled to a second constant region and the second constant region can, in some situations, be coupled to a third constant region. The light chains can each include a constant region and a variable region. The constant region of the light chains can indicate a class for the light chains. For example, the light chain of an antibody can be associated with a κ class or a λ class in mammals. More information about antibodies can be found in Schroeder H W, Cavacini L. Structure and Function of Immunoglobulins. The Journal of allergy and clinical immunology. 2010; 125(2 0 2): S41-S52. doi:10.1016/j.jaci.2009.09.046, which is incorporated by reference herein in its entirety.

The base protein 102 can include one or more base protein properties 104. The base protein properties 104 can include physical properties of the base protein 102, chemical properties of the base protein 102, or combinations thereof. The base protein properties 104 can indicate thermal stability of the base protein 102, chemical stability of the base protein 102, pH sensitivity of the base protein 102, or combinations thereof. In certain situations, the thermal stability of the base protein 102 can be indicated by a measurement of changes in the Gibbs free energy of the base protein 102. Additionally, the thermal stability of the base protein 102 can be indicated by a temperature at which the base protein 102 unfolds. The chemical stability of the base protein 102 can be indicated by the formation of certain secondary structures of the base protein 102. The base protein properties 104 can be determined by utilizing one or more assays directed to the measurement of particular properties of the base protein 102.

At 106, variants of the base protein 102 can be expressed and modified proteins 108 can be produced. The modified proteins 108 can be expressed by synthesizing deoxyribonucleic acid (DNA) sequences that encode the variations of the base protein 102 that are to be expressed. For example, to modify an amino acid at a particular position of the base protein 102, a DNA sequence encoding the base protein 102 can be modified at a region that encodes for the amino acid at the particular position such that a different amino acid is expressed when the DNA region is transcribed to messenger ribonucleic acid (mRNA) and, subsequently, translated to the modified protein. The DNA that encodes the modified proteins 108 can be placed in a host that is contained in an expression medium. In some situations, the expression medium can include a solution and the host can include a cell, such as a mammalian cell or a bacterial cell. The DNA that encodes the modified proteins 108 can be added to the host using a vector. The vector can include a plasmid or other DNA sequence into which the DNA of the modified proteins 108 can be inserted. After the protein has been expressed, purification techniques can be utilized in order to retrieve the modified proteins 108 from the expression medium. Techniques for the expression of proteins, such as antibodies, are discussed in Frenzel A, Hust M, Schirrmann T. Expression of Recombinant Antibodies. Frontiers in Immunology. 2013; 4:217. doi:10.3389/fimmu.2013.00217, which is incorporated by reference herein in its entirety.

In the illustrative implementation of FIG. 1, the modified proteins 108 can include a first modified protein 110 having a first substitution 112, a second substitution 114, and a third substitution 116. In addition, the modified proteins 108 include a second modified protein 118 having the second substitution 114 and the third substitution 116. Further, the modified proteins 108 include a third modified protein 120 having the first substitution 112, the third substitution 116, and a fourth substitution 122. The substitutions 112, 114, 116, 122 represent changes of amino acids at certain positions of the base protein 102. The squares shown in the illustrative example of FIG. 1 that represent the substitutions 112, 114, 116, 122 are not to scale and may cover multiple positions of the base protein 102; however, the squares corresponding to the substitutions 112, 114, 116, 122 are merely for illustrative purposes and are meant to indicate the specific substitutions 112, 114, 116, 122.

After the modified proteins 108 have been expressed, at 124, the values of the modified protein properties 126 can be determined. In some implementations, the values of the modified protein properties 126 can be determined using techniques similar to those utilized to determine the values of the base protein properties 104. That is, the assays utilized to determine values of certain properties of the base protein 102 can also be utilized to determine the values of modified protein properties 126. In particular implementations, the values of the modified protein properties 126 can be obtained by performing one or more analytical techniques with respect to the modified proteins 108.

The environment 100 includes a protein analysis system 128 that analyzes data corresponding to the modified proteins 108 with respect to data corresponding to the base protein 102. The protein analysis system 128 can be implemented by one or more computing devices 130. The one or more computing devices 130 can include one or more server computing devices, one or more desktop computing devices, one or more laptop computing devices, one or more tablet computing devices, one or more mobile computing devices, or combinations thereof. In certain implementations, at least a portion of the one or more computing devices 130 can be implemented in a distributed computing environment. For example, at least a portion of the one or more computing devices 130 can be implemented in a cloud computing architecture.

In some cases, the protein analysis system 128 can determine whether substitutions made to the base protein 102 produce an effect with respect to certain properties of the base protein 102. In particular implementations, the protein analysis system 128 can analyze the values of modified protein properties 126 with respect to the values of the base protein properties 104 to identify substitutions of amino acids at various positions of the base protein 102 that impact the values of one or more properties of the base protein 102. In certain situations, the protein analysis system 128 can identify substitutions to the amino acid sequence of the base protein 102 that impact properties of the modified proteins 108 in a way that improves the chemical stability of the modified proteins 108 with respect to the base protein 102. The protein analysis system 128 can also identify substitutions to the amino acid sequence of the base protein 102 that influence properties of the modified proteins 108 in a way that improves the thermal stability of the modified proteins 108 with respect to the base protein 102. By identifying substitutions that improve the chemical stability and/or thermal stability of the modified proteins 108 with respect to the base protein 102, the protein analysis system 128 can identify one or more modified proteins 108 that have improved yield in relation to the yield of the modified proteins 108 where the substitution was not made and/or that remain viable for a longer period of time than the modified proteins 108 where the substitution was not made under comparable environmental conditions. By determining the impact of a substitution with respect to values of the modified proteins 108 that include the substitution and that do not include the substitution, the impact of the substitution on the properties of the base protein 102 can be determined by the protein analysis system 128.

The protein analysis system 128 can utilize the values of the modified protein properties 126 and one or more candidate substitutions 132 to determine whether the candidate substitution 132 has an impact on the values of the properties of the modified proteins 108 that include the candidate substitution 132 and the values of the properties of the modified proteins 108 that do not include the candidate substitution 132. In particular, at 134, the protein analysis system 128 can utilize the values of modified protein properties 126 and the one or more candidate substitutions 132 to produce an initial data set 136. The initial data set 136 can include a first group 138 of the modified proteins 108 where the candidate substitution 132 has been made and a second group 140 of the modified proteins 108 where the candidate substitution 132 has not been made. The initial data set 136 can also include first values 142 of properties of the first group 138 and second values 144 of properties of the second group 140.

The protein analysis system 128 can produce the initial data set 136 by analyzing the amino acid sequences of the modified proteins 108 and identifying a subset of the modified proteins 108 that include the candidate substitution 132 and another subset of the modified proteins 108 that do not include the candidate substitution 132. In other implementations, the protein analysis system 128 can obtain data indicating the first group 138 of the modified proteins 108 with the candidate substitution 132 and the second group 140 of the modified proteins 108 without the candidate substitution 132. Additionally, the protein analysis system 128 can obtain data indicating the first values 142 and the second values 144. In particular implementations, the protein analysis system 128 can obtain a data file indicating the first group 138 and the second group 140. In various implementations, the data file can also include the first values 142 and the second values 144. In another example, the protein analysis system 128 can obtain data via one or more user interfaces that corresponds to the first group 138, the second group 140, the first values 142, the second values 144, or combinations thereof.

At 146, the protein analysis system 128 can produce a modified data set from the initial data set 136. For example, at 148, the protein analysis system 128 can couple each modified protein from the first group 138 with one or more of the modified proteins from the second group 140. To illustrate, the protein analysis system 128 can determine one or more of the modified proteins included in the second group 140 that correspond to an individual modified protein included in the first group 138. In particular implementations, the protein analysis system 128 can identify one or more modified proteins of the second group 140 that correspond with a modified protein of the first group 138 by analyzing the amino acid sequences of each modified protein of the first group 138 with respect to the amino acid sequences of the modified proteins of the second group 140. In certain implementations, the protein analysis system 128 can identify a modified protein of the second group 140 that has the same amino acid sequence as a modified protein of the first group 138 except for the candidate substitution 132. Thus, there is a single difference between the amino acid sequence of the modified protein of the second group 140 that does not have the candidate substitution 132 and the modified protein of the first group 138 that does have the candidate substitution 132. In these situations, the protein analysis system 128 can couple the modified protein of the second group 138 that does not have the candidate substitution 132 with the modified protein of the first group 138 that does have the candidate substitution 132.

In particular situations, the single difference between the amino acid sequence of the modified protein of the second group 140 and the modified protein of the first group 138 can refer to a single difference within a portion of the overall amino acid sequence of the proteins and the remainder of the amino acid sequences of the two proteins can be substantially identical. In an illustrative example, the single difference between the amino acid sequence of a modified protein from the first group 138 and a modified protein of the second group 140 can be in a constant region of a heavy chain or a constant region of a light chain, while the remainder of the amino acid sequences, such as the variable regions of the heavy chains and light chains of the two modified proteins are substantially identical. A degree of identity may be directed a portion of each amino acid sequence, or to the entire length of the amino acid sequence. Two or more amino acid sequences or portions of amino acid sequences that are “substantially identical” may have at least 50% identity, preferably at least 75% identity, more preferably at least 85% identity, most preferably at least 95%, or 100% identity.

In an illustrative example, the candidate substitution 132 can be the first substitution 112 and the protein analysis system 128 can place the first modified protein 110 and the third modified protein 120 in the first group 138 and the second modified protein 118 into the second group 140 based on the first modified protein 110 and the third modified protein 120 including the first substitution 112 and the second modified protein 118 not having the first substitution 112. Additionally, the protein analysis system 128 can couple the first modified protein 110 to the second modified protein 118 because the first modified protein 110 and the second modified protein 118 have the same amino acid sequence with the exception of the first substitution 112.

In various implementations, the protein analysis system 128 can determine that there are multiple differences between the amino acid sequences of the modified proteins 108 included in the first group 138 and the second group 140. In these scenarios, the protein analysis system 128 can couple a modified protein of the first group 138 having the candidate substitution 132 with one or more modified proteins of the second group 140 that do not have the candidate substitution 132 in a way that minimizes the differences between the amino acid sequences of individual modified proteins of the first group 138 with the amino acid sequences of the modified proteins of the second group 140. For example, the protein analysis system 128 can determine a minimum number of differences between the amino acid sequences of an individual modified protein of the first group 138 and one or more modified proteins of the second group 140. To illustrate, the protein analysis system 128 can determine that there are two differences between the amino acid sequences of an individual modified protein of the first group 138 and at least one modified protein of the second group 140. In these situations, the protein analysis system 128 can couple the individual modified protein of the first group 138 with the at least one modified protein of the second group 140. In certain scenarios, an individual modified protein of the first group 138 can be coupled with multiple modified proteins of the second group 140. In a particular example, the protein analysis system 128 can determine that a minimum number of differences between the amino acid sequences of a modified protein of the first group 138 and two modified proteins of the second group 140 is two and, consequently, the protein analysis system 128 can couple the individual modified protein of the first group 138 with the two modified proteins from the second group 140.

In certain implementations, the protein analysis system 114 can determine that a modified protein of the second group 140 has a minimum number of differences with multiple modified proteins of the first group 138. For example, the protein analysis system 128 can determine that a minimum number of differences between a first modified protein of the first group 138 and a modified protein of the second group 140 is two and that a minimum number of differences between a second modified protein of the first group 138 and the modified protein of the second group 140 is three. The protein analysis system 128 can then couple the modified protein of the second group 140 to the first modified protein of the first group 138 and to the second modified protein of the first group 138. In some implementations, other modified proteins of the second group 140 can also be coupled with the first modified protein of the first group 138 and the second modified protein of the second group 140.

After the protein analysis system 128 has produced the modified data set by coupling each modified protein of the first group 138 with one or more modified proteins of the second group 140, at 150, the protein analysis system 128 can analyze the modified data set. Analyzing the modified data set can include, at 152, determining differences between values of the properties of the individual modified proteins of the first group 138 and the respective modified proteins of the second group 140 that are coupled to the individual proteins of the first group 138. In situations where a single modified protein of the second group 140 is coupled with a modified protein of the first group 138, the protein analysis system 128 can determine a difference between values of one or more properties of the modified proteins. In an illustrative example, the first modified protein 110 can be coupled with the second modified protein 118 and the protein analysis system 128 can parse the first values 142 to identify a value for a temperature at which the first modified protein 110 unfolds and parse the second values 144 to identify a value for a temperature at which the second modified protein 118 unfolds. The protein analysis system 128 can then determine a difference between the temperature at which the first modified protein 110 unfolds and the temperature at which the second modified protein 118 unfolds. The protein analysis system 128 can proceed, for each pair of modified proteins that are coupled together from the first group 138 and the second group 140 and for each property included in the first values 142 and the second values 144, to determine the differences between the values for the particular properties.

In situations where multiple modified proteins from the second group 140 are coupled to a single variant property of the first group 138, the values for the modified proteins of the second group 140 can be combined before finding a difference between the values for the properties of the modified protein of the first group 138 and the values for the properties of the modified proteins of the second group 140 that are coupled. In some implementations, the protein analysis system 128 can determine an average of the second values 144 for the modified proteins of the second group 140 that are coupled with a modified protein of the first group 138. The protein analysis system 128 can then determine a difference between the average of the value of a property of the modified proteins of the second group 140 and the value of property of the modified protein of the first group 140. In other situations, the protein analysis system 128 can determine a median of the values for a property of modified proteins of the second group 140 that are coupled with a modified protein of the first group 138. The protein analysis system 128 can then determine a difference between the median of the values for a property of the modified proteins of the second group 140 and a value of the property for the modified protein of the first group 138.

The modified data set produced by the protein analysis system 128 can correspond to a corrected version of the initial data set 136. That is, since there are situations where there is not a single modified protein from the second group 140 that is coupled with an individual modified protein of the first group 138, the protein analysis system 128 produces a substitute modified protein that has a value for a particular property that corresponds to a combination of values of the property for multiple modified proteins of the second group 140. In this way, the protein analysis system 128 can analyze an unbalanced data set by producing a modified data set that can be analyzed in a manner similar to that of a balanced data set while minimizing the effects of analyzing the unbalanced data set.

Analyzing the modified data set can also include, at 154, determining one or more effects of the candidate substitution 132 on properties of the modified proteins 108. In some examples, the protein analysis system 128 can determine certain properties of the modified proteins 108 that are impacted by the candidate substitution 132. In certain implementations, the protein analysis system 128 can determine that the candidate substitution 132 has an effect on one or more properties of the modified proteins 108 based on the differences between the values of the one or more properties of the modified proteins 108 where the candidate substitution 132 has taken place and the values of the one or more properties for the modified proteins 108 where the candidate substitution 132 has taken place. To illustrate, the protein analysis system 128 can, for proteins of the first group 138 that have been coupled with proteins of the second group 140, analyze differences between the values for a property of the coupled modified proteins to determine if there is statistical significance between the differences of the values. In situations where there is a statistically significant difference between the values of a property for modified proteins from the first group 138 and the second group 140 that have been coupled, the protein analysis system 128 can determine that the candidate substitution 132 has an effect on the values of the property.

In various implementations, the one or more properties on which the proposed substitution 118 has an effect can impact the production of the modified proteins 108 and/or influence the stability of the modified proteins 108. For example, the protein analysis system 128 can identify one or more properties of the modified proteins 108 that increase the yield of the modified proteins 108. In another example, the protein analysis system 128 can identify one or more properties of the modified proteins 108 that improve the stability of the modified proteins at relatively higher temperatures. That is, the protein analysis system 128 can identify one or more properties of the modified proteins 108 that increase the temperature at which at least a portion of the modified proteins 108 unfold. In an additional example, the protein analysis system 128 can identify one or more properties of the modified proteins 108 that decrease the pH at which at least a portion of the modified proteins 108 are soluble. In these situations, the protein analysis system 138 can determine the properties of the modified proteins 108 that are impacted by the candidate substitution 132 and then identify the impacts of the candidate substitution 132 on the yield and/or stability of the modified proteins 108 based on the particular properties that are influenced by the candidate substitution 132.

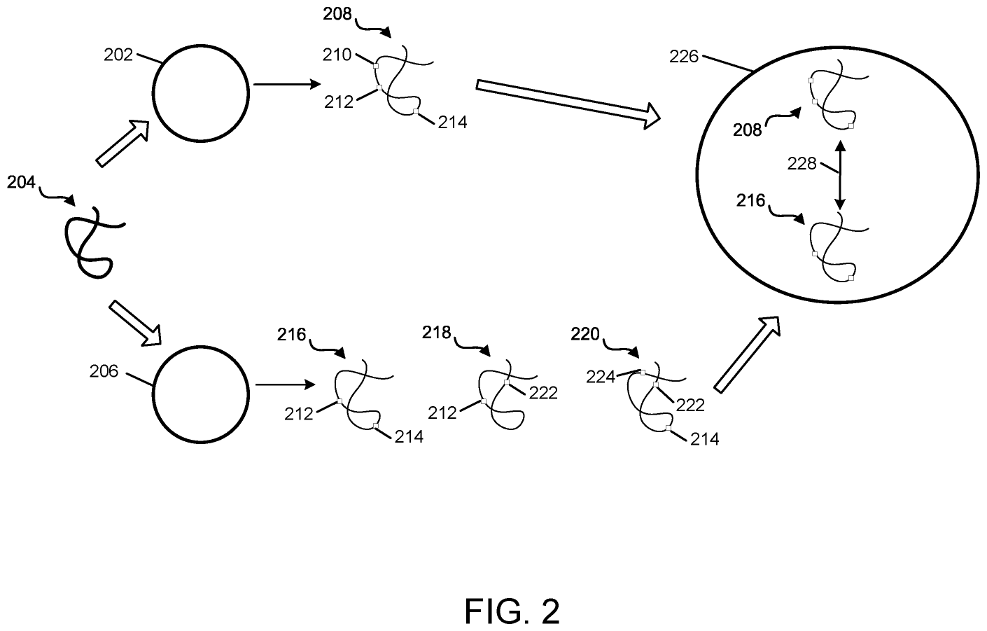

FIG. 2 illustrates some implementations of techniques to couple a modified protein having a candidate substitution to an amino acid sequence of a base protein with a single modified protein that does not include the candidate substitution. In particular, the implementations of the techniques described with respect to FIG. 2 can be utilized to identify a modified protein without a substitution of interest to couple with an individual modified protein with the substitution of interest in order to determine the effects of the substitution of interest on certain properties of the modified proteins.

In particular, a first group of modified proteins 202 can have a candidate substitution of a particular amino acid at a specified position in relation to the sequence of a base protein 204. Additionally, a second group of modified proteins 206 can include proteins that do not have the candidate substitution. Even though the second group of modified proteins 206 does not have the candidate substitution, the second group of modified proteins 206 can have other substitutions. In one example, the base protein 204 can be an antibody and a modified protein included in the first group of modified proteins 202 can have a substitution at position 35 of a constant region of a heavy chain where serine is changed to leucine.

In the illustrative example of FIG. 2, a first modified protein 208 from the first group of modified proteins 202 can include multiple substitutions with respect to the base protein 204 with at least a portion of the substitutions indicated by squares placed at various positions of the first modified protein 208. For example, the first modified protein 208 can have a first substitution 210, a second substitution 212, and a third substitution 214. Additionally, the illustrative example of FIG. 2 includes a second modified protein 216, a third modified protein 218, and a fourth modified protein 220 included in the second group of modified proteins 206 that do not have a substitution of interest. The second modified protein 218 can also have the second substitution 212 and the third substitution 214, but does not have the first substitution 208. In these situations, the first substitution 208 can be the substitution of interest that is being evaluated to identify any effects that the substitution of interest has on properties of the modified proteins. Additionally, the third modified protein 218 has the second substitution 212 and a fourth substitution 222, but does not include the first substitution 210 and the third substitution 214. Further, the fourth modified protein 220 includes the third substitution 212, the fourth substitution 222, and a fifth substitution 224, but does not include the first substitution 210 and the second substitution 212.

The substitutions 210, 212, 214, 222, 224 represent changes of amino acids at certain positions of the base protein 204. The squares shown in the illustrative example of FIG. 2 that represent the substitutions 210, 212, 214, 222, 224 are not to scale and may cover multiple positions of the base protein 204; however, the squares corresponding to the substitutions 210, 212, 214, 222, 224 are merely for illustrative purposes and are meant to indicate the specific substitutions 210, 212, 214, 222, 224.

To determine whether a substitution of interest has an effect on properties of modified proteins, each modified protein included in the first group 202 can be coupled with one or more modified proteins included in the second group 206. The proteins from the first group 202 can be coupled with the one or more proteins of the second group 206 based at least partly on a number of differences between the amino acid sequences of the modified proteins of the first group 202 and the amino acid sequences of the modified proteins of the second group 206. In particular implementations, the amino acid sequences of the individual modified proteins of the first group 202 can be compared to at least a portion of the amino acid sequences of the individual modified proteins of the second group 206. Each modified protein of the first group 202 can be coupled with one or more modified proteins of the second group 206 that have a minimum number of differences between the amino acid sequence of a respective modified protein of the first group 202 and the amino acid sequences of one or more of the modified proteins of the second group 206.

In the illustrative example of FIG. 2, the amino acid sequence of the first modified protein 208 can be compared with the amino acid sequences of the second modified protein 216, the third modified protein 218, and the fourth modified protein 220. The comparison between the amino acid sequence of the first modified protein 208 with the amino acid sequences of the modified proteins 216, 218, 220 can determine that there is one difference between the amino acid sequence of the first modified protein 208 and the amino acid sequence of the second modified protein 216, two differences between the amino acid sequence of the first modified protein 208 and the amino acid sequence of the third modified protein 218, and three differences between the amino acid sequence of the first modified protein 208 and the amino acid sequence of the fourth modified protein 220. In these situations, the first modified protein 208 can be coupled with the second modified protein 216 based at least partly on the amino acid sequence of the second modified protein 216 having a minimum number of differences with amino acid sequence of the first modified protein 208 in relation to the amino acid sequences of the third modified protein 218 and the fourth modified protein 220. In a similar manner, the other modified proteins of the first group 202 can be coupled with one or more modified proteins of the second group 204 to produce a data set 226 of coupled modified proteins. The illustrative example of FIG. 2 shows a coupling 228 that includes the first modified protein 208 and the second modified protein 216. Values of properties of the modified protein couplings included in the data set 226 can be analyzed to determine whether a substitution of interest has an impact on the properties of the modified proteins included in the first group 202 with respect to the properties of the modified proteins included in the second group 206.

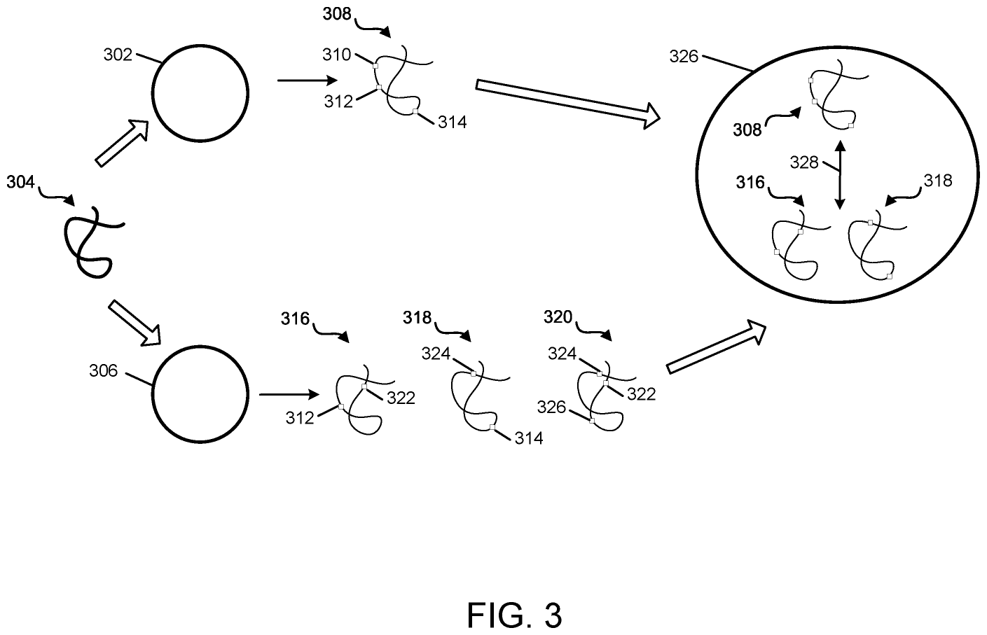

FIG. 3 illustrates some implementations of techniques to couple a modified protein having a candidate substitution to an amino acid sequence of a base protein with multiple modified proteins that do not include the candidate substitution. The features included in the illustrative example of FIG. 3 differ from those of the illustrative example of FIG. 2 in that the features included in the illustrative example of FIG. 3 correspond to coupling a pair of modified proteins where the substitution at a particular position can represent a single difference between the amino acid sequences of the pair of modified proteins, whereas the features included in the illustrative example of FIG. 3 correspond to coupling a single modified protein having a substitution of interest to a plurality of modified proteins that do not have the substitution. In these situations, there can be multiple differences at different positions between the sequence of the single modified protein having the substitution of interest and the plurality of modified proteins.

In particular, the illustrative example of FIG. 3 includes a first group of modified proteins 302 that has proteins with a substitution of a particular amino acid at a specified position in relation to the sequence of a base protein 304. The illustrative example of FIG. 3 also includes a second group of modified proteins 306 that has proteins that do not include a substitution of interest. In one example, the base protein 304 can be an antibody and a modified protein can have a substitution at position 72 of a constant region of a light chain where tyrosine is changed to histidine. The modified proteins included in the second group of modified proteins 306 can include other substitutions, but may not include a particular substitution that is being evaluated.

In the illustrative example of FIG. 3, a first modified protein 308 from the first group of modified proteins 302 can include multiple substitutions with respect to the base protein 304 with at least a portion of the substitutions indicated by squares placed at various positions of the first modified protein 308. For example, the first modified protein 308 can have a first substitution 310, a second substitution 312, and a third substitution 314. The illustrative example of FIG. 3 can also include a second modified protein 316, a third modified protein 318, and a fourth modified protein 320. The modified proteins 316, 318, 320 can be included in the second group of modified proteins 306 that do not have a substitution of interest. The second modified protein 316 can also have the second substitution 312 and a fourth substitution 322, but does not have the first substitution 310. In these situations, the first substitution 310 can be the substitution of interest that is being evaluated to identify any effects that the substitution of interest has on properties of the modified proteins. Additionally, the third modified protein 318 has the third substitution 314 and a fifth substitution 324, but does not include the first substitution 310 and the second substitution 312. Further, the fourth modified protein 318 includes the fourth substitution 322, the fifth substitution 324, and a sixth substitution 326, but does not include the first substitution 310, the second substitution 312, or the third substitution 314.

The substitutions 310, 312, 314, 322, 324, 326 represent changes of amino acids at certain positions of the base protein 304. The squares shown in the illustrative example of FIG. 3 that represent the substitutions 310, 312, 314, 322, 324, 326 are not to scale and may cover multiple positions of the base protein 204; however, the squares corresponding to the substitutions 310, 312, 314, 322, 324, 326 are merely for illustrative purposes and are meant to indicate the specific substitutions 310, 312, 314, 322, 324, 326.

To determine whether a substitution of interest has an effect on properties of modified proteins, each modified protein included in the first group 302 can be coupled with one or more modified proteins included in the second group 306. The proteins from the first group 302 can be coupled with the one or more proteins of the second group 306 based at least partly on a number of differences between the amino acid sequences of the modified proteins of the first group 302 and the amino acid sequences of the modified proteins of the second group 306. In particular implementations, the amino acid sequences of the individual modified proteins of the first group 302 can be compared to at least a portion of the amino acid sequences of the individual modified proteins of the second group 306. Each modified protein of the first group 302 can be coupled with one or more modified proteins of the second group 306 that have a minimum number of differences between the amino acid sequence of a respective modified protein of the first group 302 and the amino acid sequences of one or more of the modified proteins of the second group 306.

In the illustrative example of FIG. 3, the amino acid sequence of the first modified protein 308 can be compared with the amino acid sequences of the second modified protein 316, the third modified protein 318, and the fourth modified protein 320. The comparison between the amino acid sequence of the first modified protein 308 with the amino acid sequences of the modified proteins 316, 318, 320 can determine that there are two differences between the amino acid sequence of the first modified protein 308 and the amino acid sequences of the second modified protein 316 and the third modified protein 318 and three differences between the amino acid sequence of the first modified protein 308 and the amino acid sequence of the fourth modified protein 320. In these situations, the first modified protein 308 can be coupled with the second modified protein 316 and the third modified protein 318 based at least partly on the amino acid sequences of the second modified protein 316 and the third modified protein 318 having a minimum number of differences. In a similar manner, the other modified proteins of the first group 302 can be coupled with one or more modified proteins of the second group 306 to produce a data set 326 of coupled modified proteins. Values of properties of the modified protein couplings included in the data set 326 can be analyzed to determine whether a substitution of interest has an effect on the properties of the modified proteins included in the first group 302 with respect to the properties of the modified proteins included in the second group 304.

The illustrative example of FIG. 3 shows a coupling 328 that includes the first modified protein 308 coupled with the second modified protein 316 and the third modified protein 318. Values of properties of the modified protein couplings included in the data set 326 can be analyzed to determine whether a substitution of interest has an impact on the properties of the modified proteins included in the first group 302 with respect to the properties of the modified proteins included in the second group 306.

Since multiple modified proteins of the second group 306, that is the second modified protein 316 and the third modified protein 318, are coupled with a single protein of the first group 302, that is the first modified protein 308, in the illustrative example of FIG. 3, the values of the properties of the second modified protein 316 and the third modified protein 318 can be modified in order to analyze the effect of the candidate substitution on the properties of the modified proteins. For example, before analyzing the values of the properties of the second modified protein 316 and the third modified protein 318 with respect to the values of the properties of the first modified protein 308, the values of at least a portion of the properties of the second modified protein 316 and the third modified protein 318 can be combined. To illustrate, an average of the values of a particular property for the second modified protein 316 and the third modified protein 318 can be determined before analyzing the values of the particular property of the second modified protein 316 and the third modified protein 318 in relation to the value of the particular property for the first modified protein 308.

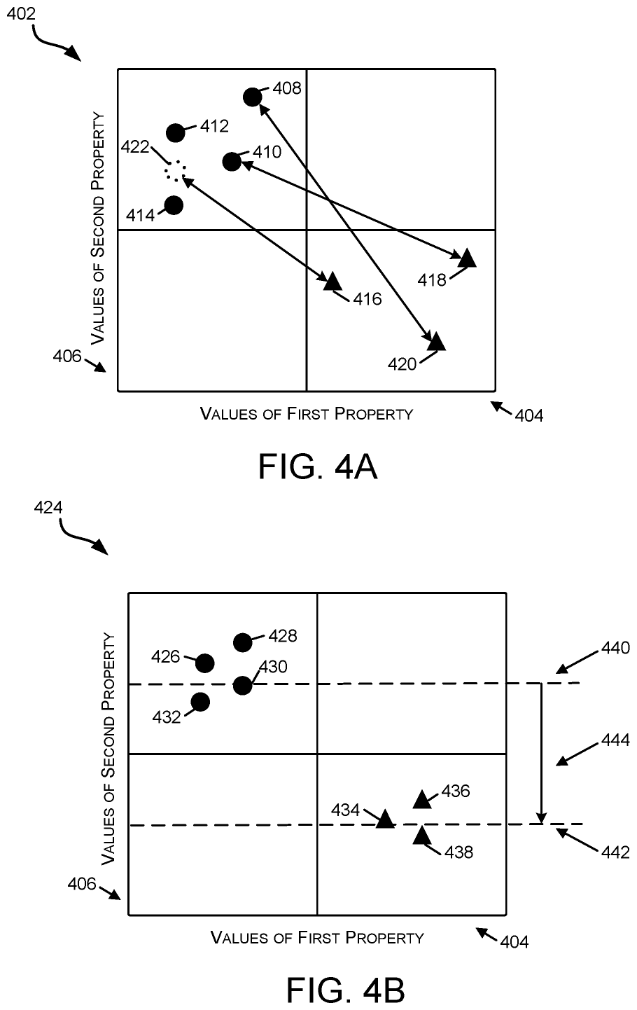

FIGS. 4A and 4B illustrate some implementations of techniques to analyze differences between values of properties of modified proteins that have a candidate substitution to an amino acid sequence of a base protein with respect to values of properties of modified proteins that do not have the candidate substitution. FIG. 4 includes a first plot 402 having an x-axis 404 that represents values of a first property and a y-axis 406 that represents values of a second property. The first property and the second property can correspond to properties of proteins. For example, the first property and the second property can include Gibbs free energy of proteins, temperatures at which proteins unfold, pHs at which proteins become insoluble, and so forth. The first plot 402 indicates the values of the first property and the values of the second property for a number of proteins. In particular, the first plot 402 indicates the values of the first property and the values of the second property for a number of proteins that have been modified to include a candidate substitution and a number of proteins that do not include the candidate substitution. In the illustrative example of FIG. 4, the values of the first property and the values of the second property for proteins that do not include the candidate substitution are represented by the circles at 408, 410, 412, and 414. Additionally, the values of the first property and the values of the second property for proteins that include the substitution of interest are represented by the triangles at 416, 418, and 420.

In certain implementations, to determine whether the candidate substitution has an effect on the first property and/or the second property, the values of the first property and the values of the second property for the proteins that include the substitution of interest and for the proteins that do not include the substitution of interest can be analyzed. In some cases, the analysis of the values of the first property and the values of the second property for the proteins that include the candidate substitution and the proteins that do not include the candidate substitution can include coupling each protein that does have the substitution of interest with one or more proteins that do not have the substitution of interest. In particular implementations, the proteins that do include the candidate substitution can be coupled with one or more proteins that do not have the candidate substitution by comparing the amino acid sequences of the proteins that do include the candidate substitution and the proteins that do not include the candidate substitution. A protein that does have the candidate substitution can be coupled with one or more proteins that do not include the candidate substitution based on identifying one or more proteins that do not have the candidate substitution and have amino acid sequences with a minimum number of differences with the amino acid sequence of the protein that does have the candidate substitution.

In the first plot 402 of FIG. 4A, the protein with the candidate substitution that is represented by triangle 418 is coupled with the protein of interest that does not include the candidate substitution represented by circle 410. Additionally, the protein with the candidate substitution that is represented by triangle 420 is coupled with the protein of interest that does not include the candidate substitution represented by circle 408. Further, the protein with the candidate substitution that is represented by the triangle 416 is not coupled with a single protein that does not have the candidate substitution. In the illustrative example of FIG. 4, the protein represented by triangle 420 is coupled with the proteins represented by the circles 412 and 414. In this situation, to facilitate the analysis to determine whether the candidate substitution has an effect on the first property and/or the second property, the values of the first property and the values of the second property for the proteins represented by the circles 412 and 414 are combined to produce values indicated by the circle 422. In particular implementations, the values of the first property and the values of the second property represented by the circle 412 and the circle 414 can be combined by determining an average of the values represented by the circles 412 and 414. Thus, in the illustrative example of FIG. 4A, the protein represented by the triangle 416 is shown as being coupled to the circle 422 representing the combination of the values of the first property and the values of the second property for the proteins represented by the circles 412 and 414. In the first plot 402, the couplings between proteins are shown by arrows between the respective triangles and circles.

After analyzing the data included in the first plot 402, a second plot 424 shown in FIG. 4B can be produced. The second plot 424 can indicate a modified set of data based on the couplings between each protein where a candidate substitution has been made and one or more proteins where the candidate substitution has not been made. In some implementations, the second plot 424 can indicate a normalized version of the initial data set shown in the first plot 402 based on the differences between the values of the first property and the values of the second property for the proteins that include the candidate substitution of interest and the proteins that do not include the candidate substitution. In the illustrative example of FIG. 4B, the second plot 424 includes circles 426, 428, 430, and 432 that represent the modified versions of the values of the first property and the modified values of the second property for the proteins indicated by circles 408, 410, 412, 414 in the initial data set shown in the first plot 402. Additionally, the second plot 424 also includes triangles 434, 436, 438 that represent the modified values of the first property and the modified values of the second property for the proteins indicated by triangles 416, 418, 420 in the initial data set shown in the first plot 402.

In particular implementations, an impact of a candidate substitution on the values of the first property and/or the values of the second property can be determined based on an analysis of the data included in the second plot 424. For example, a first mean value 440 for the values of the second property can be determined based on the values of the second property represented by the circles 426, 428, 430, 432. In addition, a second mean value 442 for the values of the second property can be determined based on the values of the second property represented by the triangles 434, 436, 438. Further, a difference 444 between the first mean value 440 and the second mean value 442 can be determined. Computational tests can be performed based on the difference 444 and the data included in the second plot 424 to determine whether the difference 444 is statistically significant. To illustrate, an analysis of variance or t-test can be performed to determine whether the difference 444 is statistically significant. In situations where the difference 444 is statistically significant, a determination can be made that the candidate substitution has an effect on the values of the second property 406 with respect to the proteins represented by the circles 408, 410, 412, 414 and the triangles 416, 418, 420 of the first plot 402.

In certain situations, where the difference 444 is statistically significant, a determination can be made as to whether the difference 444 improves the yield and/or stability of the proteins represented by the triangles 416, 418, 420 with respect to the proteins represented by the circles 408, 410, 412, 414. Whether the difference 444 improves the yield and/or stability of the proteins represented by the triangles 416, 418, 420 can depend on the property for which the difference 444 is being determined. For example, in situations where the difference 444 indicates that the temperature at which the proteins represented by the circles 408, 410, 412, 414 unfold is higher than the temperature at which the proteins represented by the circles 416, 418, 420 unfold, then the difference 444 can indicate that the candidate substitution can be detrimental to the yield and/or stability of the proteins represented by the circles 416, 418, 420. In another example, in situations where the difference 444 indicates that the pH at which the proteins represented by the circles 416, 418, 420 are stable decreases with respect to the proteins represented by the circles 408, 410, 412, 414, then the difference 444 can indicate that the substitution of interest can improve the yield and/or stability of the proteins represented by the triangles 416, 418, 420.

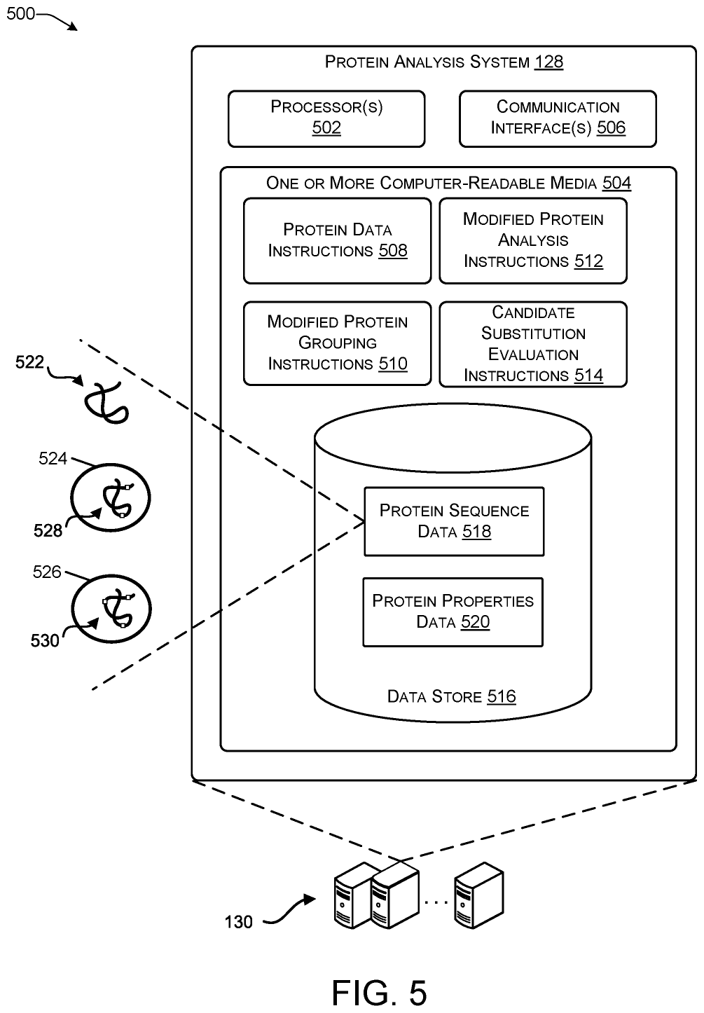

FIG. 5 illustrates some implementations of a system 500 to determine the impact on properties of proteins based on modifications to amino acid sequences of the proteins. The system 500 includes a protein analysis system 128 can be implemented by the one or more computing devices 130. In some implementations, the one or more first computing devices 130 can be included in a cloud computing architecture that operates the one or more first computing devices 130 on behalf of an entity implementing the protein analysis system 128, such as a user of the protein analysis system 128. In these scenarios, the cloud computing architecture can instantiate one or more virtual machine instances on behalf of the entity implementing the protein analysis system 128 using the one or more computing devices 130. The cloud computing architecture can be located remote from the entity implementing the protein analysis system 128. In additional implementations, the one or more computing devices 130 can be under the direct control of the entity implementing the protein analysis system 128. For example, the entity implementing the protein analysis system 128 can maintain the one or more computing devices 130 to perform operations related to analyzing substitutions of amino acid sequences of modified proteins to identify substitutions having an effect on the properties of the modified proteins. In various implementations, the one or more computing devices 130 can include one or more server computers.

The protein analysis system 128 can include one or more processors, such as processor 502. The one or more processors 502 can include at least one hardware processor, such as a microprocessor. In some cases, the one or more processors 502 can include a central processing unit (CPU), a graphics processing unit (GPU), or both a CPU and GPU, or other processing units. Additionally, the one or more processors 502 can include a local memory that may store program modules, program data, and/or one or more operating systems.

In addition, the protein analysis system 128 can include one or more computer-readable storage media, such as computer-readable storage media 504. The computer-readable storage media 504 can include volatile and nonvolatile memory and/or removable and non-removable media implemented in any type of technology for storage of information, such as computer-readable instructions, data structures, program modules, or other data. Such computer-readable storage media 504 can include, but is not limited to, RAM, ROM, EEPROM, flash memory or other memory technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, removable storage media, or any other medium that can be used to store the desired information and that can be accessed by a computing device. Depending on the configuration of the protein analysis system 114, the computer-readable storage media 504 can be a type of tangible computer-readable storage media and can be a non-transitory storage media.

The protein analysis system 128 can include one or more communication interfaces 506 to communicate with other computing devices via one or more networks (not shown), such as one or more of the Internet, a cable network, a satellite network, a wide area wireless communication network, a wired local area network, a wireless local area network, or a public switched telephone network (PSTN).

The computer-readable storage media 504 can be used to store any number of functional components that are executable by the one or more processors 502. In many implementations, these functional components comprise instructions or programs that are executable by the one or more processors 502 and that, when executed, implement operational logic for performing the operations attributed to the protein analysis system 128. Functional components of the protein analysis system 128 that can be executed on the one or more processors 502 for implementing the various functions and features related to analyzing substitutions of amino acid sequences of modified proteins to identify substitutions having an effect on the properties of the modified proteins, as described herein, include protein data instructions 508, modified protein grouping instructions 510, modified protein analysis instructions 512, and candidate substitution evaluation instructions 514.

Additionally, the one or more first computing devices 502 can include one or more input/output devices (not shown). The one or more input/output devices can include a display device, keyboard, a remote controller, a mouse, a printer, audio input/output devices, a speaker, a microphone, a camera, and so forth

The protein analysis system 128 can also include, or be coupled to, a data store 516 that can include, but is not limited to, RAM, ROM, EEPROM, flash memory, one or more hard disks, solid state drives, optical memory (e.g. CD, DVD), or other non-transient memory technologies. The data store 516 can maintain information that is utilized by the protein analysis system 128 to perform operations related to analyzing substitutions of amino acid sequences of modified proteins to identify substitutions having an impact on the properties of the modified proteins. For example, the data store 516 can store protein sequence data 518 and protein properties data 520. The protein sequence data 518 can include the amino acid sequences of base proteins and variants of the base proteins that are being analyzed by the protein analysis system 128. In some cases, the protein sequence data 518 can indicate positions of the modified proteins at which substitutions are made relative to sequences of base proteins.

In the illustrative example of FIG. 5, the protein sequence data 518 includes amino acid sequences of one or more base proteins, such as an illustrative base protein 522. Also in the illustrative example of FIG. 5, the protein sequence data 518 includes amino acid sequences for variants of the base protein 522, such as an amino acid sequences of a first group of modified proteins 524 and a second group of modified proteins 526. The first group of modified proteins 524 can include a number of proteins that are variants of the base protein 522 that include a substitution of interest, such as the illustrative first modified protein 528, and the second group of modified proteins 526 can include a number of proteins that are variants of the base protein 522 that do not include a substitution of interest, such as the illustrative second modified protein 530. The substitution of interest can be a candidate substitution of an amino acid at a particular position of the base protein, where the candidate substitution is being evaluated to determine the effect of the candidate substitution on the properties of the modified proteins.

The protein properties data 520 can include values of properties of the base proteins and the values of properties of variants of the base proteins. The properties included in the protein properties data 520 can include chemical properties of proteins, physical properties of proteins, or combinations thereof. In some examples, the protein properties data 520 can include temperatures at which proteins unfold. In another example, the protein properties data 520 can include solubility information for the proteins. To illustrate, the protein properties data 520 can include a percentage of the heavy chain molecular weight for antibodies at a given pH. The protein properties data 520 can also include additional information related to the molecular weight of the proteins, such as total molecular weight, heavy chain molecular weight, light chain molecular weight, percentage of heavy chain molecular weight relative to total molecular weight, percentage of light chain molecular weight relative to total molecular weight, or combinations thereof. The protein properties data 520 can also include Gibbs free energy of the base proteins and the variants of the base proteins. Further, the protein properties data 520 can be related to secondary structures of the base proteins and secondary structures of the variants of the base proteins. To illustrate, the protein properties data 520 can indicate locations of secondary structures of base proteins and variants of the base proteins. The protein properties data 520 can also indicate spectroscopic measurements and characteristics (e.g., peaks) that indicate the presence of certain secondary structures, such as wavelengths where secondary structures can be indicated for base proteins and variants of the base proteins.

The protein data instructions 508 can be executable by the one or more processors 502 to obtain and store data related to base proteins and variants of the base proteins. In some implementations, the protein data instructions 508 can obtain sequence data for base proteins and variants of the base proteins. The sequence data obtained using the protein data instructions 508 can be stored in the data store 516 as the protein sequence data 518. The protein data instructions 508 can also obtain data related to properties of the base proteins and the variants of the base proteins. In particular implementations, the protein data instructions 508 can obtain values for physical properties and/or chemical properties of base proteins and variants of the base proteins. The protein data instructions 508 can store the data related to properties of base proteins and variants of the base proteins as the protein properties data 520.

In various implementations, the protein data instructions 508 can obtain data by producing one or more user interfaces that include one or more user interface elements to capture data corresponding to base proteins and variants of the base proteins. In additional implementations, the protein data instructions 508 can obtain data related to base proteins and variants of the base proteins from a web site. In further implementations, the protein data instructions 508 can obtain data related to base proteins and variants of the base proteins from one or more data storage devices. The one or more data storage devices can include removable data storage devices, such as memory sticks, flash drives, or thumb drives. The one or more data storage devices can also include data stores coupled to the protein analysis system 128 via one or more networks, such as wired local area networks, wireless local area networks, wireless wide area networks, or combinations thereof.

The modified protein grouping instructions 510 can be executable by the one or more processors 502 to group modified proteins according to one or more criteria. In particular implementations, the modified protein grouping instructions 510 can group modified proteins according to a candidate substitution. The candidate substitution can correspond to a modification of an amino acid sequence of a base protein. The modified protein grouping instructions 510 can group a number of modified proteins into a first group of modified proteins that have the candidate substitution and a second group of modified proteins that do not have the candidate substitution. In certain implementations, the modified protein grouping instructions 510 can identify a subset of proteins that are then grouped according to the candidate substitution. For example, the modified protein grouping instructions 510 can identify a base protein, such as the base protein 522, and then determine variants of the base protein based at least partly on differences between the amino acid sequence of the base protein and the amino acid sequences of the modified proteins. In the illustrative example of FIG. 5, the variants of the base protein 522 can include the first group of modified proteins 524 and the second group of modified proteins 526. In some cases, the modified protein grouping instructions 510 can obtain input indicating a base protein and a group of variants of the base protein. In other situations, the modified protein grouping instructions 510 can obtain input indicating a base protein and then compare the sequence of the base protein with sequences of additional proteins included in the protein sequence data 518. In certain implementations, the modified protein grouping instructions 510 can determine that a protein is a variant of a base protein based at least partly on a threshold amount of the amino acid sequence of the protein being homologous with respect to the amino acid sequence of the base protein.

The modified protein analysis instructions 512 can be executable by the one or more processors 502 to analyze modified proteins. In some implementations, the modified protein analysis instructions 512 can analyze modified proteins with respect to one or more candidate substitutions. The one or more candidate substitutions can include substitutions made on amino acid sequences of proteins that cause differences in the amino acid sequences with respect to base proteins. The analysis of the modified proteins can be performed to determine whether or not the one or more substitutions of interest have an impact on the properties of the modified proteins. In particular implementations, the modified protein analysis instructions 512 can analyze values of properties of the modified proteins that include the one or more candidate substitutions with respect to values of properties of modified proteins that do not include the one or more candidate substitutions to determine whether the one or more candidate substitutions have an impact on the properties of the modified proteins.

In certain implementations, the modified protein analysis instructions 512 can perform an analysis of modified proteins by identifying one or more modified proteins that do not have the candidate substitution that correspond to one or more modified proteins that do include the candidate substitution. In particular implementations, the modified protein analysis instructions 512 can couple a modified protein that includes a candidate substitution with one or more modified proteins that do not include the candidate substitution. The modified protein analysis instructions 512 can then identify values for a property of modified proteins included in a first group that have a candidate substitution and identify values for the property of modified proteins included in a second group that do not include a candidate substitution. In an illustrative example, the modified protein analysis instructions 512 can couple the first modified protein 528 with the second modified protein 530. The protein analysis instructions 512 can then identify values of properties of the first modified protein 528 and analyze the values of the properties of the first modified protein 528 with respect to values of the properties of the second modified protein 530.

Analyzing the values of properties of modified proteins that have a candidate substitution with respect to values of properties of modified proteins that do not have the candidate substitution can include determining differences between the values of properties of the modified proteins that have the candidate substitution and the values of properties for modified proteins that do have the candidate substitution. Additionally, analyzing the values of properties of the modified proteins that have a candidate substitution with respect to values of properties of the modified proteins that do not have the candidate substitution can include normalizing the values of the properties based on the differences in the values. Further, in some implementations, analyzing the values of properties of modified proteins that have a candidate substitution with respect to values of properties of modified proteins that do not have the candidate substitution can include determining an average of values of properties for modified proteins that do not have the candidate substitution that are coupled with a modified protein that does have the candidate substitution. The analysis performed by the protein analysis instructions 512 can produce a data set that is different from an initial data set for the proteins that have been modified with respect to a base protein. For example, the modified data set can indicate couplings between modified proteins having a candidate substitution and one or more modified proteins that do not include the candidate substitution. Also, in situations where multiple modified proteins without a candidate substitution have been coupled with a modified protein having the candidate substitution, the modified data set can include combined values (e.g., average values) of the individual values for one or more properties of the multiple modified proteins without the candidate substitution. The modified data set can also include normalized values for the properties of the modified proteins in scenarios where values for properties of individual modified proteins without a candidate substitution have been combined.

The candidate substitution evaluation instructions 514 can perform an additional analysis of the modified data set. For example, the candidate substitution evaluation instructions 514 can determine a mean of the normalized values of the properties of the modified proteins that have the candidate substitution and a mean of the normalized values of the properties of the modified proteins that do not have the candidate substitution. The candidate substitution evaluation instructions 514 can determine a difference between the means and implement one or more statistical techniques to determine whether there is a statistically significant difference between the mean of the normalized values for the properties of the modified proteins that include the candidate substitution and the mean of the normalized values for the properties of the modified proteins that do not include the candidate substitution. In situations where there is a statistically significant difference between the means, the candidate substitution evaluation instructions 514 can determine that the candidate substitution has an influence on properties of the modified proteins. In certain implementations, a determination that a candidate substitution influences values of properties of the modified proteins can indicate a threshold probability that the candidate substitution influences values of one or more properties of the modified proteins. In particular implementations, the threshold probability can be at least a 70% probability, at least an 80% probability, at least a 90% probability, at least a 95% probability, at least a 97% probability, at least a 98% probability, or at least a 99% probability that the candidate substitution has an effect on the values of one or more properties of the modified proteins.

The influence of the candidate substitution on the values of properties of the modified proteins can be based at least partly on the particular properties that are influenced by the candidate substitution. For example, in scenarios where the candidate substitution has an effect on the temperature at which a modified protein unfolds, the candidate substitution evaluation instructions 514 can determine that the candidate substitution has an effect on the stability of the modified protein at certain temperatures. In another example, in situations where the candidate substitution has an effect on the solubility of the modified protein, the candidate substitution evaluation instructions 514 can determine that the candidate substitution has an effect on the yield of the modified protein.

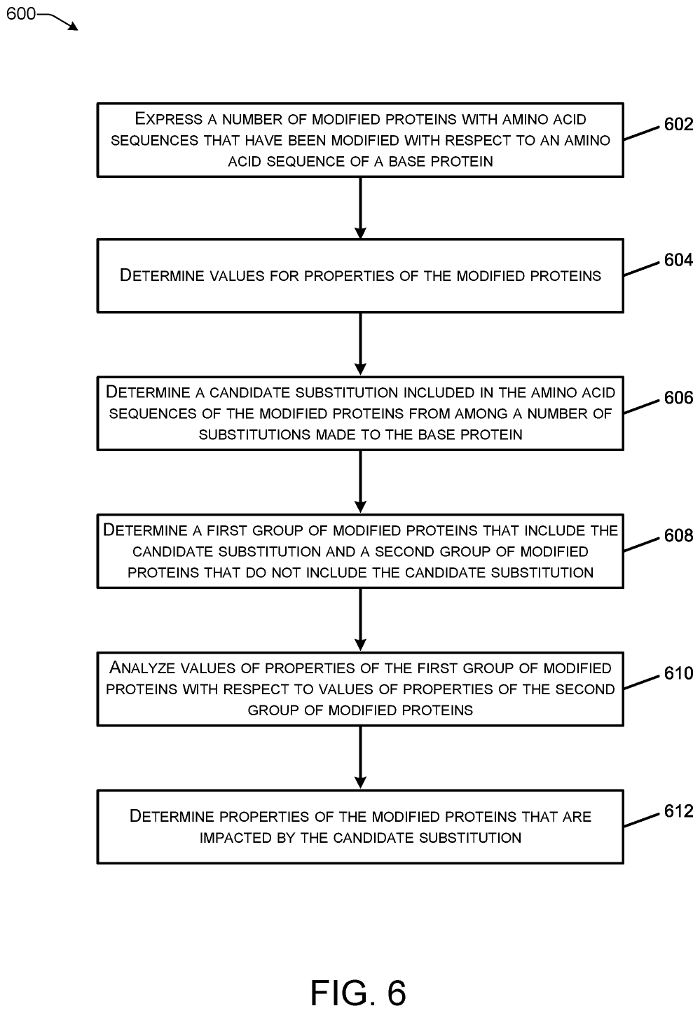

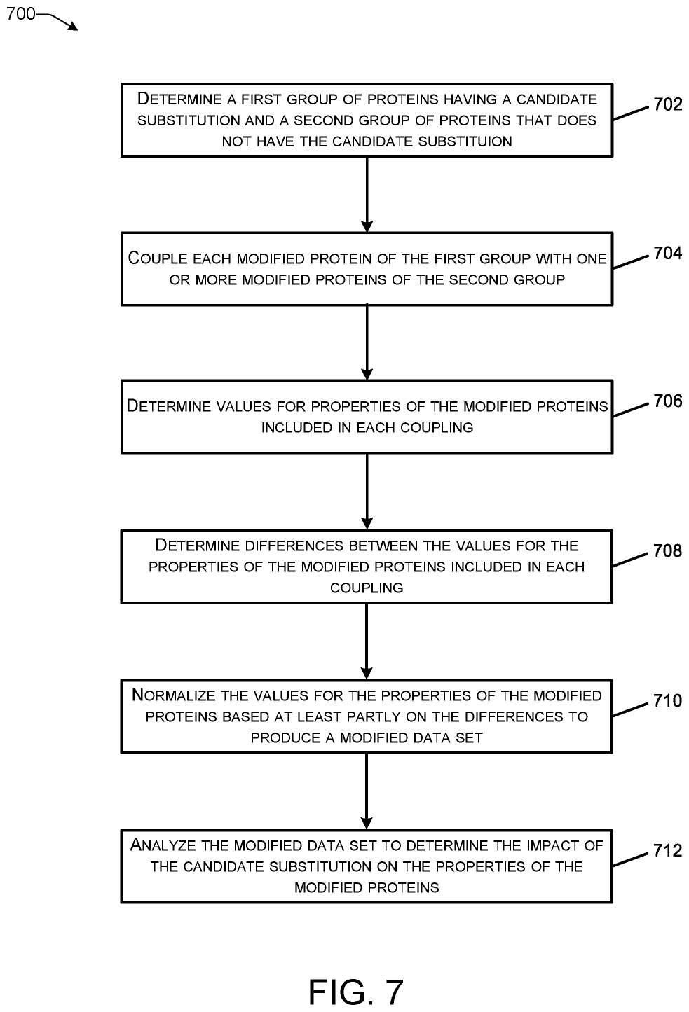

FIGS. 6 and 7 illustrate example processes of determining the impact of substitutions of amino acid sequences of base proteins on properties of the base proteins. These processes (as well as each process described herein) are illustrated as logical flow graphs, each operation of which represents a sequence of operations that can, at least in part, be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the process.

FIG. 6 is a flow diagram of a first example process 600 to identify properties of proteins that are impacted by substitutions of amino acid sequences of the proteins. At 602, the process 600 includes expressing a number of modified proteins with sequences that have been modified with respect to a sequence of a base protein. In some cases, the base protein can include an antibody. In particular implementations, the protein can include an antibody that is produced to bind with a virus. In various implementations, the changes to the amino acid sequence of the base protein can be identified and DNA of the base protein can be modified such that the modified amino acid sequences are expressed from the modified DNA. In certain implementations, the modifications to the amino acid sequence of the base protein can be part of a design of experiments to determine if substitutions to the amino acid sequence of the base protein impact the properties of the base protein and/or the properties of the modified proteins.

At 604, the process 600 includes determining values for properties of the modified proteins. After the modified proteins have been expressed, the values for the properties of the modified proteins can be determined by performing analytical techniques with respect to the expressed proteins. The analytical techniques can be performed, in some cases, using one or more assays. Additionally, at 606, the process 600 includes determining a candidate substitution included in the amino acid sequences of the modified proteins from among a number of substitutions made to the base protein. In particular implementations, the candidate substitution can be obtained by a system that analyzes the effects of substitutions of amino acid sequences, such as the protein analysis system 128 of FIG. 1 and FIG. 5, via one or more user interface elements. In some implementations, the candidate substitution can be identified from a design of experiments utilized to determine the modifications made to the base protein.

At 608, the process 600 includes determining a first group of modified proteins that include the candidate substitution and a second group of modified proteins that do not include the candidate substitution. In particular implementations, the first group of modified proteins and the second group of modified proteins can be determined by comparing amino acid sequences of at least a portion of the modified proteins with the amino acid sequence of the base protein with respect to the candidate substitution. In other implementations, the first group of modified proteins and the second group of modified proteins can be identified in a data file provided to a protein analysis system or specified via one or more user interface elements.

At 610, the process 600 includes analyzing values of properties of the first group of modified proteins with respect to values of properties of the second group of modified proteins. For example, differences between values of properties of the first group of modified proteins and values of properties of the second group of modified proteins can be determined. In some implementations, individual modified proteins of the first group can be coupled with one or more modified proteins of the second group to perform the analysis. To illustrate, a modified protein of the first group can be coupled with one or more modified proteins of the second group that have a minimal number of differences between the amino acid sequences of the modified protein of the first group and the one or more modified proteins of the second group. By determining differences between values of properties for only the modified proteins that have been coupled, the processing resources and memory resources utilized to analyze the values of the properties of the first group and the second group are minimized because the values for properties of every modified protein of the first group are not analyzed with respect to values for properties of every modified protein of the second group. Instead, values for properties of each modified protein of the first group are analyzed with respect values of properties for a subset of the modified proteins of the second group.

At 612, the process 600 includes determining properties of the modified proteins that are impacted by the candidate substitution. In particular implementations, differences between values of properties of modified proteins of the first group and values of modified proteins of the second group can indicate properties that are impacted by the candidate substitution. For example, a determination can be made that a property is impacted by the candidate substitution by determining that the differences between the values for the properties of the first group and values for the properties of the second group are statistically significant.

FIG. 7 is a flow diagram of a second example process 700 to identify properties of proteins that are impacted by substitutions of amino acid sequences of the proteins. At 702, the process 700 includes determining a first group of proteins having a candidate substitution and a second group of proteins that does not have the candidate substitution. At 704, the process 700 includes coupling each modified protein of the first group with one or more modified proteins of the second group. For example, a protein that includes the candidate substitution can be coupled with one or more candidates that do not have the candidate substitution based at least partly on the amino acid sequences of the one or more candidates that do not have the candidate substitution having a minimal number of differences with the amino acid sequence of the protein that does include the candidate substitution.

At 706, the process 700 includes determining values for properties of the modified proteins included in each coupling. In situations where a single protein from the first group is coupled with a single protein from the second group, the values for the properties of the modified proteins included in the coupling can be obtained from a data store that includes the values for the properties or obtained via one or more user interface elements. In scenarios where a single protein from the first group is coupled with multiple proteins from the second group, the values for the properties of the proteins from the second group can be modified. For example, an average of the values for the properties of the individual proteins in the second group can be determined to provide single values for each of the properties that can be compared with the values for the properties of the single protein included in the first group.

At 708, the process 700 includes determining differences between the values for the properties of the modified proteins included in each coupling and, at 710, the process 700 includes normalizing the values for the properties of the modified proteins based at least partly on the differences to produce a modified data set. In particular implementations, each coupling of proteins from the first group and the second group can have values for each property being evaluated determined such that a single value of the protein of the first group included in the coupling can be compared with a single value of the one or more proteins of the second group included in the coupling. In this way, a single difference value can be determined for each coupling. The original data set can then be modified based on these difference values to produce the modified data set. That is, the values of properties from an original data set can be modified to correct for situations where multiple proteins of the second group have been coupled with a single protein of the first group. In certain implementations, the modified data set can represent an approximation of a balanced data set that is produced from an unbalanced data set.

At 712, the process 700 can include analyzing the modified data set to determine the impact of the candidate substitution on the properties of the modified proteins. In some implementations, a mean for the modified values for a property of the first group can be compared with a mean for the modified values for the property of the second group. The difference between the means can indicate a statistical significance of the candidate substitution with respect to the property. Depending on the property for which the candidate substitution has an effect, additional determinations can be made regarding the impact of the candidate substitution. For example, a determination can be made that yield of a protein having the candidate substitution increases based on determining that a property indicating solubility of the protein improves under certain conditions, such as a lower pH.

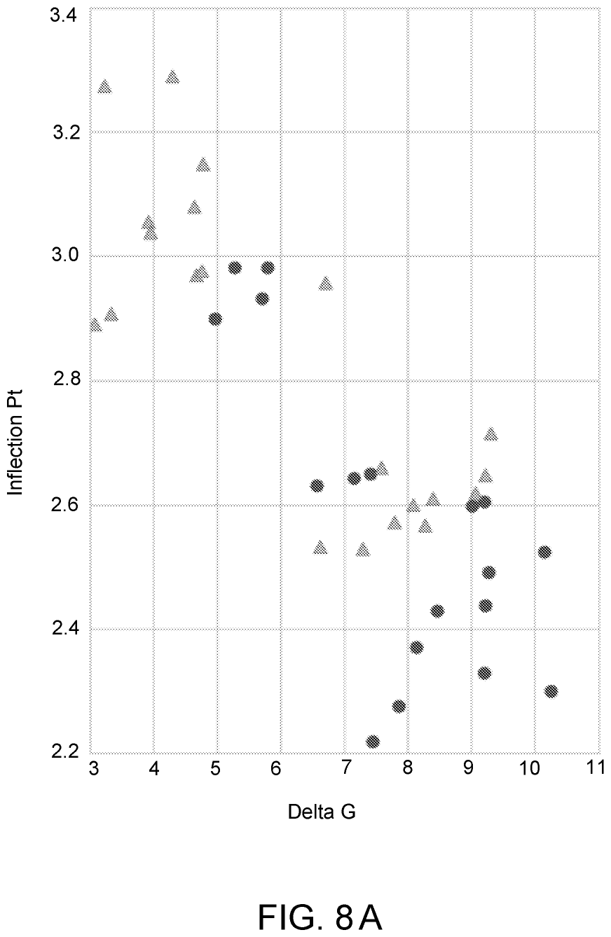

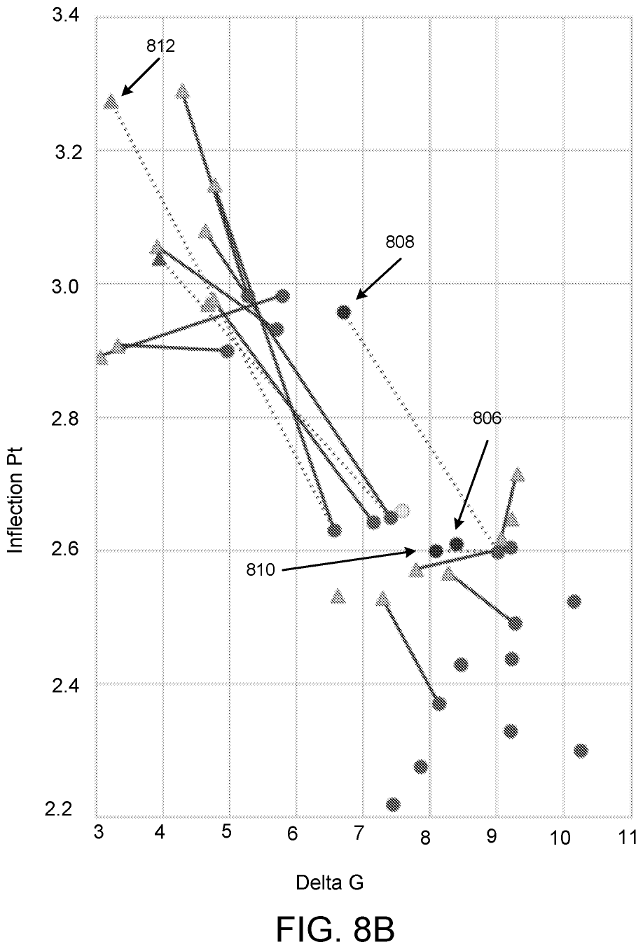

FIG. 8A illustrates a first plot 802 of values of properties of proteins that have been modified to include a particular substitution with respect to a base protein and values of properties of proteins that have not been modified to include the particular substitution and FIG. 8B illustrates a second plot 804 showing couplings between proteins that have been modified to include the particular substitution and proteins that have not been modified to include the particular substitution. In particular, the plots 802, 804 shows values for properties of proteins that include a candidate substitution represented by triangles and values for properties of proteins that do not include a candidate substitution represented by circles. The x-axis of the plots 802, 804 indicate values for Gibbs free energy and the y-axis of the plots 802, 804 indicate the amount of denaturant required to make the proteins unfold.

In FIG. 8B, the red lines, both dotted, dashed, and solid represent couplings between proteins that have the candidate substitution and one or more proteins that do not have the candidate substitution. In some cases, additional symbols have been added at 806, 808, 810, and 812 when multiple proteins that do not include the candidate substitution have been coupled with a single protein that does include the candidate substitution. The values represented by the additional symbols 806, 808, 810, 812 can represent the averages for the values of the properties shown in the second plot 804 for multiple proteins that do not include the candidate substitution that have been coupled with a single protein that does include the candidate substitution. Symbols colored other than green or blue and those connected by dashed lines, indicate proteins that are coupled, but contain one or more changes other than the candidate substitution and are therefore less accurate references.

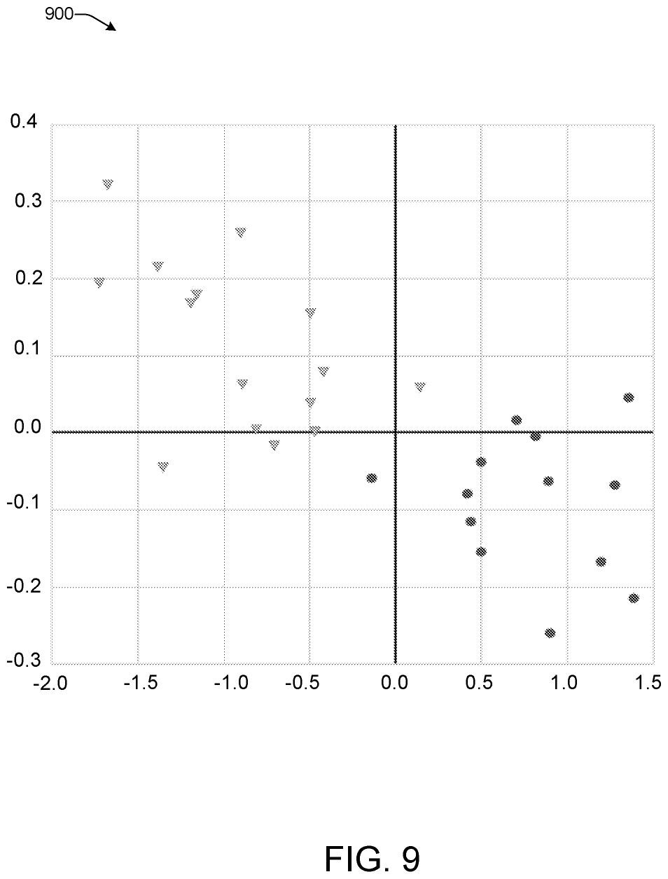

FIG. 9 illustrates a plot 900 that shows the data points of the first plot of FIG. 8 modified based on the couplings shown in the second plot of FIG. 8. In particular, the plot 900 is produced by normalizing the values for the properties of the proteins that include the candidate substitution with respect to the proteins that do not include the candidate substitution based on the differences between the proteins that have been coupled with each other shown in the second plot 804 of FIG. 8. This demonstrates how the values for proteins shown in FIG. 8A are insufficient to determine significance of effect on their own, but when coupled and adjusted to produce FIG. 9, the significance of the effect on the two properties is clear.

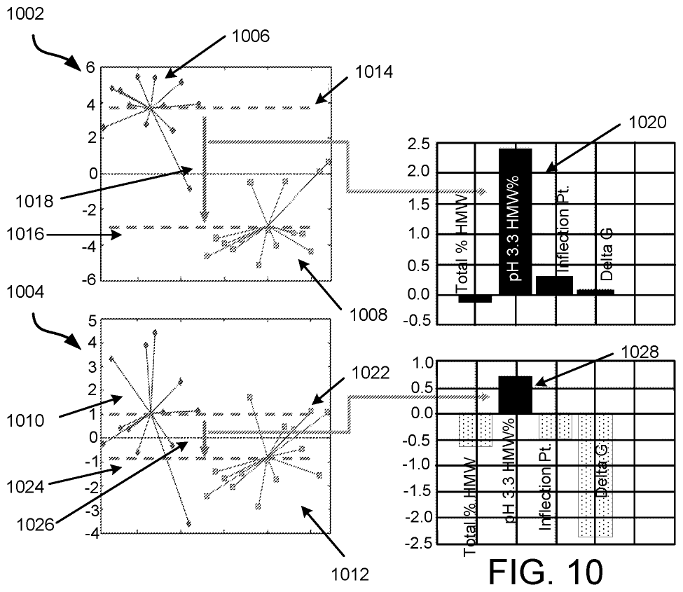

FIG. 10 illustrates a first plot 1002 showing a difference in means between a first group of data 1006 derived from proteins that have a first candidate substitution and a second group of data 1008 derived from proteins that do not have the first candidate substitution and a second plot 1004 showing a difference in means between a first group of data 1010 derived from proteins that have a second candidate substitution and a second group of data 1012 derived from proteins that do not have the second candidate substitution. In the illustrative example of FIG. 10, the first candidate substitution can be associated with the antibody under study having a modification at position 2 of a heavy chain from valine to serine. Additionally, the second candidate substitution can be associated with the antibody having a modification at position 3 of a light chain from threonine to lysine. The first plot 1002 and the second plot 1004 can represent normalized data sets for values of at least one property for the proteins included in the first groups 1006, 1010 and the second groups 1008, 1012.

The first plot 1002 includes a first mean 1014 for the values for the property of the first group of proteins 1006 and a second mean 1016 for the values for the property of the second group of proteins 1008. Additionally, the first plot 1002 includes a difference 1018 between the first mean 1014 and the second mean 1016. Further, the illustrative example of FIG. 10 shows that the difference 1018 corresponds to a bar 1020 indicating the difference 1018 with respect to a particular property, the percentage of heavy chain molecular weight with respect to the total makeup of the proteins at a pH of 3.3. Furthermore, the shading of the bar 1020 can indicate that the difference 1018 is statistically significant.

In addition, the second plot 1004 includes a first mean 1022 for the values for the property of the first group of proteins 1010 and a second mean 1024 for the values for the property of the second group of proteins 1012. Additionally, the second plot 1004 includes a difference 1026 between the first mean 1022 and the second mean 1024. Further, the illustrative example of FIG. 10 shows that the difference 1024 corresponds to a bar 1028 indicating the difference 1024 with respect to a particular property, the percentage of heavy chain molecular weight with respect to the total molecular weight of the proteins at a pH of 3.3. The shading of the bar 1028 can indicate that the difference 1016 is statistically significant.

### Example Implementations

Clause 1. A method comprising: expressing a number of proteins with amino acid sequences that have been modified at a number of positions with respect to an amino acid sequence of a base protein; determining values for properties of the number of proteins; determining a candidate substitution indicating a difference between amino acid sequences of a portion of the number of proteins and the amino acid sequence of the base protein at a particular position; determining a first group of the number of proteins that includes the candidate substitution and a second group of the number of proteins that does not include the candidate substitution; performing an analysis of first values for the properties of the first group with respect to second values for the properties of the second group; and determining, based at least partly on the analysis, that the candidate substitution produces a statistically significant effect on values of at least one property of the first group.

Clause 2. The method of clause 1, further comprising determining that at least one of yield or stability increases based at least partly on the statistically significant effect produced by the candidate substitution.

Clause 3. The method of clause 1 or 2, wherein the analysis includes: coupling a first protein of the first group with a second protein and a third protein of the second group; determining an average value for the at least one property based on a first value for the at least one property of the second protein and a second value for the at least one property of the third protein; and determining a difference between the average value and an additional value for the property of the first protein.

Clause 4. The method of clause 3, further comprising: performing a comparison between a first amino acid sequence of the first protein and amino acid sequences of the second group; and determining, based at least partly on the comparison, that differences between the first amino acid sequence and a second amino acid sequence of the second protein and a third amino acid sequence of the third protein are less than a threshold number.

Clause 5. The method of any of clauses 1-4, wherein the base protein is an antibody.

Clause 6. The method of any one of clauses 1-5, wherein a value for a property of a protein of the number of proteins is determined by performing one or more assays with respect to the protein.

Clause 7. The method of any one of clauses 1-6, wherein determining the values for the properties of the number of proteins includes at least one of: determining a value of a temperature at which a protein of the number of proteins unfolds to an extent that the protein is unable to bind a target molecule for the protein; determining a value of a pH at which the protein becomes insoluble in water; or determining a percentage of heavy chain molecular weight with respect to total molecular weight for the protein.

Clause 8. The method of any one of clauses 1-7, wherein the candidate substitution is one of a plurality of substitutions made in the amino acid sequences of the first group of the number of proteins with respect to the amino acid sequence of the base protein.

Clause 9. The method of any one of clauses 1-8, wherein determining that the candidate substitution produces the statistically significant effect on the values of the at least one property of the first group includes performing an analysis of variance or a t-test.

Clause 10. A method comprising: determining that a first group of proteins has a candidate substitution and that a second group of proteins does not have the candidate substitution; coupling a protein included in the first group with a plurality of proteins included in the second group; determining a value for a property with respect to the protein; determining additional values for the property with respect to individual proteins of the plurality of proteins; determining, based at least partly on the additional values, an average value for the property with respect to the plurality of proteins; determining a difference between the value for the property and the average value for the property; and determining, based at least partly on the difference, an amount of impact of the candidate substitution on the property.

Clause 11. The method of clause 10, further comprising: comparing an amino acid sequence of the protein with individual amino acid sequences of proteins included in the second group of proteins; determining a minimum number of differences between the amino acid sequence of the protein and the individual amino acid sequences of the proteins included in the second group of proteins.

Clause 12. The method of clause 11, further comprising: determining a first number of differences between an amino acid sequence of the protein and a first additional amino acid sequence of a first additional protein included in the second group; determining a second number of differences between the amino acid sequence of the protein and a second additional amino acid sequence of a second additional protein included in the second group, the second number of differences being different than the first number of differences; determining that the first number of differences corresponds to the minimum number of differences; determining that the second number of differences is greater than the minimum number of differences; and adding the first additional protein to the plurality of proteins.

Clause 13. The method of any one of clauses 10-12, wherein determining the amount of impact of the candidate substitution on the property includes determining a probability that the candidate substitution has an impact on the property.

Clause 14. The method of any one of clauses 10-13, further comprising: generating a user interface that includes at least one user interface element to capture the value for the property with respect to the protein.

Clause 15. The method of any one of clauses 10-14, further comprising: obtaining the value for the property with respect to the protein from a web site or from a data storage device.

Clause 16. The method of any one of clauses 10-15, wherein the property includes a temperature at which at least a portion of the protein begins to unfold, and the method further comprising: determining, based at least partly on the amount of impact of the candidate substitution on the property, that the candidate substitution has an effect on stability of the protein.

Clause 17. The method of any one of clauses 10-16, wherein the property includes solubility of the protein, and the method further comprises: determining, based at least partly on the amount of impact on the candidate substitution on the property, that the candidate substitution has an effect on yield of the protein.

Clause 18. The method of any one of clauses 10-17, wherein the property includes Gibbs free energy, total molecular weight, heavy chain molecular weight, light chain molecular weight, percentage of heavy chain molecular weight relative to total molecular weight, percentage of light chain molecular weight relative to total molecular weight, or presence of a secondary structure.

Clause 19. The method of any one of clauses 10-18, wherein the candidate substitution includes an amino acid at a particular position of the protein that is different from an additional amino acid at a same position of a base protein.

Clause 20. The method of clause 19, wherein the protein has a plurality of additional substitutions at a plurality of additional positions with respect to amino acids at the plurality of additional positions of the base protein.

Clause 21. A system comprising: one or more processors; and one or more non-transitory computer-readable media storing computer-readable instructions that, when executed by the one or more processors, perform operations comprising: determining a candidate substitution indicating a difference between amino acid sequences of a portion of a number of proteins and an amino acid sequence of a base protein at a particular position, wherein the number of proteins include amino acid sequences that have been modified at a number of positions with respect to the amino acid sequence of the base protein; determining values of properties of the number of proteins; determining a first group of the number of proteins that includes the candidate substitution and a second group of the number of proteins that does not include the candidate substitution; performing an analysis of first values for the properties of the first group with respect to second values for the properties of the second group; and determining, based at least partly on the analysis, that the candidate substitution produces a statistically significant effect on values of at least one property of the first group.

Clause 22. The system of clause 21, wherein the operations further comprise determining that at least one of yield or stability increases based at least partly on the statistically significant effect produced by the candidate substitution.

Clause 23. The system of clause 21 or 22, wherein the analysis includes: coupling a first protein of the first group with a second protein and a third protein of the second group; determining an average value for the at least one property based on a first value for the at least one property of the second protein and a second value for the at least one property of the third protein; and determining a difference between the average value and an additional value for the property of the first protein.

Clause 24. The system of clause 23, wherein the operations further comprise: performing a comparison between a first amino acid sequence of the first protein and amino acid sequences of the second group; and determining, based at least partly on the comparison, that differences between the first amino acid sequence and a second amino acid sequence of the second protein and a third amino acid sequence of the third protein are less than a threshold number.

Clause 25. The system of any of clauses 21-24, wherein the base protein is an antibody.

Clause 26. The system of any one of clauses 21-25, wherein a value for a property of a protein of the number of proteins is determined by performing one or more assays with respect to the protein.

Clause 27. The system of any one of clauses 21-26, wherein determining the values for the properties of the number of proteins includes at least one of: determining a value of a temperature at which a protein of the number of proteins unfolds to an extent that the protein is unable to bind a target molecule for the protein; determining a value of a pH at which the protein becomes insoluble in water; or determining a percentage of heavy chain molecular weight with respect to total molecular weight for the protein.

Clause 28. The system of any one of clauses 21-27, wherein the candidate substitution is one of a plurality of substitutions made in the amino acid sequences of the first group of the number of proteins with respect to the amino acid sequence of the base protein.

Clause 29. The system of any one of clauses 21-28, wherein determining that the candidate substitution produces the statistically significant effect on the values of the at least one property of the first group includes performing an analysis of variance or a t-test.

Clause 30. A system comprising: one or more processors; and one or more non-transitory computer-readable media storing computer-readable instructions that, when executed by the one or more processors, perform operations comprising: determining that a first group of proteins has a candidate substitution and that a second group of proteins does not have the candidate substitution; coupling a protein included in the first group with a plurality of proteins included in the second group; determining a value for a property with respect to the protein; determining additional values for the property with respect to individual proteins of the plurality of proteins; determining, based at least partly on the additional values, an average value for the property with respect to the plurality of proteins; determining a difference between the value for the property and the average value for the property; and determining, based at least partly on the difference, an amount of impact of the candidate substitution on the property.

Clause 31. The system of clause 30, wherein the operations further comprise: comparing an amino acid sequence of the protein with individual amino acid sequences of proteins included in the second group of proteins; determining a minimum number of differences between the amino acid sequence of the protein and the individual amino acid sequences of the proteins included in the second group of proteins.

Clause 32. The system of clause 31, wherein the operations further comprise: determining a first number of differences between an amino acid sequence of the protein and a first additional amino acid sequence of a first additional protein included in the second group; determining a second number of differences between the amino acid sequence of the protein and a second additional amino acid sequence of a second additional protein included in the second group, the second number of differences being different than the first number of differences; determining that the first number of differences corresponds to the minimum number of differences; determining that the second number of differences is greater than the minimum number of differences; and adding the first additional protein to the plurality of proteins.

Clause 33. The system of any one of clauses 30-32, wherein determining the amount of impact of the candidate substitution on the property includes determining a probability that the candidate substitution has an impact on the property.

Clause 34. The system of any one of clauses 30-33, wherein the operations further comprise: generating a user interface that includes at least one user interface element to capture the value for the property with respect to the protein.

Clause 35. The system of any one of clauses 30-34, wherein the operations further comprise: obtaining the value for the property with respect to the protein from a web site or from a data storage device.

Clause 36. The system of any one of clauses 30-35, wherein the property includes a temperature at which at least a portion of the protein begins to unfold, and the operations further comprise: determining, based at least partly on the amount of impact of the candidate substitution on the property, that the candidate substitution has an effect on stability of the protein.

Clause 37. The system of any one of clauses 30-36, wherein the property includes solubility of the protein, and the operations further comprise: determining, based at least partly on the amount of impact on the candidate substitution on the property, that the candidate substitution has an effect on yield of the protein.

Clause 38. The system of any one of clauses 30-37, wherein the property includes Gibbs free energy, total molecular weight, heavy chain molecular weight, light chain molecular weight, percentage of heavy chain molecular weight relative to total molecular weight, percentage of light chain molecular weight relative to total molecular weight, or presence of a secondary structure.

Clause 39. The system of any one of clauses 30-38, wherein the candidate substitution includes an amino acid at a particular position of the protein that is different from an additional amino acid at a same position of a base protein.

Clause 40. The system of clause 39, wherein the protein has a plurality of additional substitutions at a plurality of additional positions with respect to amino acids at the plurality of additional positions of the base protein.

The subject matter described above is provided by way of illustration only and should not be construed as limiting. Furthermore, the claimed subject matter is not limited to implementations that solve any or all disadvantages noted in any part of this disclosure. Various modifications and changes can be made to the subject matter described herein without following the example configurations and applications illustrated and described, and without departing from the true spirit and scope of the present invention, which is set forth in the following claims.

1. A method comprising:
expressing a number of proteins with amino acid sequences that have, been modified at a number of positions with respect to an amino acid sequence of a base protein;
determining values for a plurality of properties of the number of proteins;
determining a candidate substitution indicating a difference between amino acid sequences of a portion of the number of proteins and the amino acid sequence of the base protein at a respective position;
determining a first group of the number of proteins that includes the candidate substitution and a second group of the number of proteins that does not include the candidate substitution;
coupling a first protein of the first group with a second protein of the second group and a third protein of the second group;
determining an average value for a property of the plurality of properties based on a first value for the property of the second protein and a second value for the property of the third protein;
determining a difference between the average value and an additional value for the property of the first protein
and
determining, based at least partly on the difference, that the candidate substitution produces a statistically significant effect on values of at least one property of the first group.

2. The method of claim 1, further comprising determining that at least one of yield or stability increases based at least partly on the statistically significant effect produced by the candidate substitution.

3. (canceled)

4. The method of claim 1, further comprising:
performing a comparison between a first amino acid sequence of the first protein and amino acid sequences of the second group; and
determining, based at least partly on the comparison, that differences between the first amino acid sequence and a second amino acid sequence of the second protein and a third amino acid sequence of the third protein are less than a threshold number.

5. (canceled)

6. The method of claim 1, wherein a value for the property of at least one of the first protein, the second protein, or the third protein is determined by performing one or more assays.

7. The method of claim 1, wherein determining the values for the plurality of properties of the number of proteins includes at least one of:
determining a value of a temperature at which the first protein unfolds to an extent that the first protein is unable to bind a target molecule for the first protein;
determining a value of a pH at which the first protein becomes insoluble in water; or
determining a percentage of heavy chain molecular weight with respect to total molecular weight for the first protein.

8. The method of claim 1, wherein the candidate substitution is one of a plurality of substitutions made in the amino acid sequences of the first group of the number of proteins with respect to the amino acid sequence of the base protein.

9. The method of claim 1, wherein determining that the candidate substitution produces the statistically significant effect on the values of the at least one property of the first group includes performing an analysis of variance or a t-test.

10. A method comprising:
determining that a first group of proteins has a candidate substitution and that a second group of proteins does not have the candidate substitution;
coupling a protein included in the first group with a plurality of proteins included in the second group;
determining a value for a property with respect to the protein;
determining additional values for the property with respect to individual proteins of the plurality of proteins;
determining, based at least partly on the additional values, an average value for the property with respect to the plurality of proteins;
determining a difference between the value, or the property and the average value for the property; and
determining, based at least partly on the difference, an amount of impact of the candidate substitution on the property.

11. The method of claim 10, further comprising:
comparing an amino acid sequence of the protein with individual amino acid sequences of proteins included in the second group of proteins;
determining a minimum number of differences between the amino acid sequence of the protein and the individual amino acid sequences of the proteins included in the second group of proteins.

12. The method of claim 11, further comprising:
determining a first number of differences between the amino acid sequence of the protein and a first additional amino acid sequence of a first additional protein included in the second group;
determining a second number of differences between the amino acid sequence of the protein and a second additional amino acid sequence of a second additional protein included in the second group, the second number of differences being different than the first number of differences;
determining that the first number of differences corresponds to the minimum number of differences;
determining that the second number of differences is greater than the minimum number of differences; and
adding the first additional protein to the plurality of proteins.

13. The method of claim 10, wherein determining the amount of impact of the candidate substitution on the property includes determining a probability that the candidate substitution has an impact on the property.

14. The method of claim 10, further comprising:
generating a user interface that includes at least one user interface element to capture the value for the property with respect to the protein.

15. (canceled)

16. The method of claim 10, wherein the property includes a temperature at which at least a portion of the protein begins to unfold, and the method further comprises:
determining, based at least partly on the amount of impact of the candidate substitution on the property, that the candidate substitution has an effect on stability of the protein.

17. The method of claim 10, wherein the property includes solubility of the protein, and the method further comprises:
determining, based at least partly on the amount of impact on the candidate substitution on the property, that the candidate substitution has an effect on yield of the protein.

18. The method of claim 10, wherein the property includes Gibbs free energy, total molecular weight, heavy chain molecular weight, light chain molecular weight, percentage of heavy chain molecular weight relative to total molecular weight, percentage of light chain molecular weight relative to total molecular weight, or presence of a secondary structure.

19. The method of claim 10, wherein the candidate substitution includes an amino acid at a respective position of the protein that is different from an additional amino acid at a same position of a base protein.

20. The method of claim 19, wherein the protein has a plurality of additional substitutions at a plurality of additional positions with respect to amino acids at the plurality of additional positions of the base protein.

21. A system comprising:
one or n processors; and
one or more non-transitory computer-readable media storing computer-readable instructions that, when executed by the one or more processors, perform operations comprising:
determining a candidate substitution indicating a difference between amino acid sequences of a portion of a number of proteins and an amino acid sequence of a base protein at a respective position, wherein the number of proteins include amino acid sequences that have been modified at a number of positions with respect to the amino acid sequence of the base protein;
determining values of properties of the number of proteins;
determining first group of the number of proteins that includes the candidate substitution and a second group of the number of proteins that does not include the candidate substitution;
coupling a first protein of the first group with a second protein of the second group and a third protein of the second group;
determining an average value for the at least one property based on a first value for the at least one property of the second protein and a second value for the at least one property of the third protein;
determining a difference between the average value and an additional value for the property of the first protein; and
determining, based at least partly on the difference between the average value and an additional value for the property of the first protein, that the candidate substitution produces a statistically significant effect on values of at least one property of the first group.

22. The system of claim 21, wherein the one or more non-transitory computer-readable media store additional computer-readable instructions that, when executed by the one or more processors, perform additional operations comprising:
comparing an amino acid sequence of the first protein with individual amino acid sequences of proteins included in the second group of the number of proteins;
determining a minimum number of differences between the amino acid sequence of the first protein and the individual amino acid sequences of the proteins included in the second group of the number of proteins.

23. The system of claim 22, wherein the one or more non-transitory computer-readable media store additional computer-readable instructions that, when executed by the one or more processors, perform additional operations comprising:
determining a first number of differences between the amino acid sequence of the first protein and a first additional amino acid sequence of a first additional protein included in the second group of the number of proteins;
determining a second number of differences between the amino acid sequence of the first protein and a second additional amino acid sequence of a second additional protein included in the second group of the number of proteins, the second number of differences being different than the first number of differences;
determining that the first number of differences corresponds to the minimum number of differences;
determining that the second number of differences is greater than the minimum number of differences; and
adding the first additional protein to a plurality of proteins coupled to the first protein.
